(19) United States
(12) Patent Application Publication
(10) Pub. No.: US 2015/0275302 A1
(43) Pub. Date: Oct. 1, 2015

(54) DETERMINATION OF SINGLE NUCLEOTIDE POLYMORPHISMS USEFUL TO PREDICT RESPONSE FOR RASAGILINE

(71) Applicants: Mario Masellis, Thornhill (CA); Joanne Knight, Toronto (CA); Maureen Shannon Collinson, Toronto (CA); Anthony Edward Lang, Toronto (CA); James Lowery Kennedy, Toronto (CA); Joseph Levy, Kfar-Saba (IL); Amir Tchelet, Hod Hasharon (IL); Iris Grossman, Yakir (IL); Eli Eyal, Petach-Tikva (IL); Ofra Barnett, Haifa (IL)

(72) Inventors: Mario Masellis, Thornhill (CA); Joanne Knight, Toronto (CA); Maureen Shannon Collinson, Toronto (CA); Anthony Edward Lang, Toronto (CA); James Lowery Kennedy, Toronto (CA); Joseph Levy, Kfar-Saba (IL); Amir Tchelet, Hod Hasharon (IL); Iris Grossman, Yakir (IL); Eli Eyal, Petach-Tikva (IL); Ofra Barnett, Haifa (IL)

(73) Assignee: TEVA PHARMACEUTICAL INDUSTRIES, LTD., Petach-Tikva (IL)

(21) Appl. No.: 14/674,606

(22) Filed: Mar. 31, 2015

Related U.S. Application Data
(60) Provisional application No. 61/973,603, filed on Apr. 1, 2014.

(51) Int. Cl. C12Q 1/68; A61K 45/06; A61K 31/4985; A61K 31/48; A61K 31/198; A61K 31/4045; A61K 31/428; A61K 31/381; A61K 31/135; A61K 31/439

(52) U.S. Cl. CPC C12Q 1/6883; A61K 31/135; A61K 45/06; A61K 31/4985; A61K 31/439; A61K 31/198; A61K 31/4045; A61K 31/428; A61K 31/381; A61K 31/48; C12Q 2600/156; C12Q 2600/106

(57) ABSTRACT

This application provides a method for treating a human subject afflicted with Parkinson's disease (PD) with a pharmaceutical composition comprising rasagiline or a pharmaceutically acceptable salt of rasagiline, and a pharmaceutically acceptable carrier, comprising the steps of:
(i) obtaining a biological sample comprising a genome from the human subject afflicted with Parkinson's disease;
(ii) assaying the DNA or RNA of the biological sample from the human subject using a probe or a primer, to determine the diploid genotype of the human subject at single nucleotide polymorphism (SNP) rs1076560 or rs2283265;
(iii) identifying the human subject as a predicted responder to rasagiline if the diploid genotype is CC at rs1076560, CC at rs2283265, or CC at both rs1076560 and rs2283265; and
(iv) administering the pharmaceutical composition comprising rasagiline and a pharmaceutically acceptable carrier to the human subject if the human subject is identified as a predicted responder to rasagiline.

This application claims benefit of U.S. Provisional Application No. 61/973,603, filed Apr. 1, 2014, the entire content of which is hereby incorporated by reference herein.

Throughout this application various publications, published patent applications, and patents are referenced. The disclosures of these documents in their entireties are hereby incorporated by reference into this application in order to more fully describe the state of the art to which this invention pertains.

### BACKGROUND OF THE INVENTION

### Rasagiline

U.S. Pat. Nos. 5,532,415, 5,387,612, 5,453,446, 5,457,133, 5,599,991, 5,744,500, 5,891,923, 5,668,181, 5,576,353, 5,519,061, 5,786,390, 6,316,504, 6,630,514, 7,750,051, and 7,855,233 disclose R(+)-N-propargyl-l-aminoindan (“R-PAI”), also known as rasagiline, and its pharmaceutically acceptable salts. These U.S. patents also disclose that rasagiline is a selective inhibitor of the B-form of the enzyme monoamine oxidase (“MAO-B”) and is useful in treating Parkinson's disease and various other conditions by inhibition of MAO-B in the brain.

U.S. Pat. Nos. 6,126,968, 7,572,834, and 7,598,420, U.S. patent application Ser. Nos. 12/283,022, and 12/283,107 and PCT publications WO 95/11016 and WO 2006/014973, hereby incorporated by reference, disclose pharmaceutical compositions comprising rasagiline and processes for their preparation.

AZILECT® is a commercially available rasagiline mesylate immediate release formulation indicated for the treatment of the signs and symptoms of idiopathic Parkinson's disease as initial monotherapy and as adjunct therapy to levodopa. The current marketed formulation of rasagiline (Azilect®) is rapidly absorbed, reaching peak plasma concentration (tₘₐₓ) in approximately 1 hour. The absolute bioavailability of rasagiline is about 36%. (AZILECT® Product Label, May 2006).

### Pharmacogenomics

Pharmacogenomics is the methodology which associates genetic variability with physiological and clinical responses to drug. Pharmacogenetics is a subset of pharmacogenomics and is defined as “the study of variations in DNA sequence as related to drug response” (ICH E15; http://www.fda.gov/downloads/RegulatoryInformation/Guidances/ucm129296.pdf). Pharmacogenetics often focuses on genetic polymorphisms in genes related to drug metabolism, drug mechanism of action, underlying disease type, and drug associated side effects. Pharmacogenetics is the cornerstone of Personalized Medicine which allows the development of individualized drug therapies to obtain effective and safe treatment, as well as to adjust existing treatment regimens to further optimize the efficacy and safety profile for the individual patient.

Pharmacogenetics has become a core component of many drug development programs, being used to explain variability in drug response among subjects in clinical trials, to address unexpected emerging clinical issues, such as adverse events, to determine eligibility for a clinical trial (pre-screening) to optimize trial yield, to develop drug companion diagnostic tests to identify patients who are more likely or less likely to benefit from treatment or who may be at risk of adverse events, to provide information in drug labels to guide physician treatment decisions, to better understand the mechanism of action or metabolism of new and existing drugs, and to provide better understanding of disease mechanisms as associated with treatment response.

Generally, Pharmacogenetics analyses are performed in either of two methodology approaches: Candidate genes research technique, and Genome Wide Association Study (GWAS). Candidate genes research technique is a hypothesis driven approach, based on the detection of polymorphisms in candidate genes pre-selected using knowledge of the disease, the drug's mode of action, toxicology or metabolism of the drug. The Genome Wide Association Study (GWAS) screens a standard, known set of more than 1 M (one million) polymorphisms across the entire genome. This approach is used when related genes are unknown or novel ones with small effect sizes are being sought, given sufficient size of the cohorts tested. DNA arrays used for GWAS can be also analyzed per gene as in the candidate gene approach, but often do not cover functional or non-SNP variation. Furthermore, only tag SNPs are used on GWAS microarrays and therefore important variation within some candidate genes may be missed depending on how densely covered a genomic region is with tag SNPs.

### SUMMARY OF THE INVENTION

This invention provides a method for treating a human subject afflicted with Parkinson's disease (PD) with a pharmaceutical composition comprising rasagiline or a pharmaceutically acceptable salt of rasagiline, and a pharmaceutically acceptable carrier, comprising the steps of:
(i) obtaining a biological sample comprising a genome from the human subject afflicted with Parkinson's disease;
(ii) assaying the DNA or RNA of the biological sample from the human subject using probes or primers, to determine the diploid genotype of the human subject at single nucleotide polymorphism (SNP) rs1076560 or rs2283265;
(iii) identifying the human subject as a predicted responder to rasagiline if the diploid genotype is CC at rs1076560, CC at rs2283265, or CC at both rs1076560 and rs2283265; and
(iv) administering the pharmaceutical composition comprising rasagiline and a pharmaceutically acceptable carrier to the human subject if the human subject is identified as a predicted responder to rasagiline.

This invention also provides a method for treating a human subject afflicted with Parkinson's disease comprising the steps of:
(i) administering to the human subject a therapeutic amount of a pharmaceutical composition comprising rasagiline, or a pharmaceutically acceptable salt of rasagiline, and a pharmaceutically acceptable carrier;
(ii) obtaining a biological sample comprising a genome from the human subject afflicted with Parkinson's disease;
(iii) assaying the DNA or RNA of the biological sample from the human subject using probes or primers, to determine the diploid genotype of the human subject at single nucleotide polymorphism (SNP) rs1076560 or rs2283265;
(iv) identifying the human subject as a predicted responder to rasagiline if the diploid genotype is CC at rs1076560, CC at rs2283265, or CC at both rs1076560 and rs2283265; and
(v) continuing administration of the pharmaceutical composition if the human subject is identified as a predicted responder to rasagiline, or modifying the administration of the pharmaceutical composition to the human subject if the human subject is not identified as a predicted responder to rasagiline.

This invention also provides a diagnostic kit for evaluating responsiveness to treatment with rasagiline in a human subject afflicted with Parkinson's disease, the kit comprising
(i) at least one probe specific for SNP rs1076560 or rs2283265, and
(ii) instructions for use of the at least one probe to evaluate responsiveness of the subject to treatment with rasagiline.

This invention also provides a diagnostic kit for evaluating responsiveness to treatment with rasagiline in a human subject afflicted with Parkinson's disease, the kit comprising
(i) at least one pair of PCR primers designed to amplify one or more DNA segments which include SNP rs1076560 or rs2283265, and
(ii) instructions for use of the at least one pair of PCR primers to evaluate responsiveness of the subject to treatment with rasagiline.

This invention also provides a PCR amplification kit comprising
(i) at least one pair of PCR primers designed to amplify one or more DNA segments which include SNP rs1076560 or rs2283265, and
(ii) instructions for use of the PCR primers to amplify the one or more segments of DNA.

This invention also provides a diagnostic kit for evaluating responsiveness to treatment with rasagiline in a human subject afflicted with Parkinson's disease, the kit comprising
(i) a reagent for performing restriction fragment length polymorphism (RFLP) analysis, sequencing, single strand conformation polymorphism analysis (SSCP), chemical cleavage of mismatch (CCM), gene chip, denaturing high performance liquid chromatography (DHPLC) and polymerase chain reaction (PCR) amplification for determining the identity of one or more SNPs wherein the one or more SNPs comprises at least one of rs1076560 or rs2283265, and
   instructions for use of the reagent to evaluate responsiveness of the subject to treatment with rasagiline.

This invention also provides a method of determining the identity of the alleles of fewer than 10000 single nucleotide polymorphisms (SNPs) in a subject selected from the group of subjects consisting of human subjects diagnosed with Parkinson's disease to produce a polymorphic profile of the selected subject diagnosed with Parkinson's disease, comprising
(i) obtaining a biological sample comprising a genome from the selected subject diagnosed with Parkinson's disease;
(ii) selecting for allelic identity analysis at least a SNP located at rs1076560 and a SNP located at rs2283265 within the genome of the selected subject diagnosed with Parkinson's disease; and
(iii) assaying, with probes or primers, whether
   a) the allelic identity at rs1076560 is CC within the nucleotide sequence of the genome in the biological sample of step i), and
   b) the allelic identity at rs2283265 is CC within the nucleotide sequence of the genome in the biological sample of step i), and
wherein fewer than 10000 SNPs are selected for allelic identity analysis in step ii) and the same fewer than 10000 SNPs are assayed in step iii).
This invention also provides a physical or electronic database comprising the polymorphic profiles of human subjects afflicted with PD, wherein each polymorphic profile includes the diploid genotype of fewer than 10000 SNPs, and the fewer than 10000 SNPs include rs1076560 and rs36023.

### BRIEF DESCRIPTION OF THE DRAWINGS

FIG. 1. Ancestry Clustering. Principal Component Analysis for determining ethnicity of the studied cohort. There were not enough markers to determine ancestry sub-clustering correctly, therefore the self-reported ethnicity, known to serve as a good proxy, was used.

FIG. 2. Heterzygosity distribution. Heterozygosity plot for the pharmacogenetic population after removing duplicate samples and those of non-Caucasian ancestry.

FIG. 3. Treatment group residuals. Residuals vs Fitted values for the fixed effect model for the treatment group of individuals. No specific pattern detected, thus normality was assumed.

FIG. 4. Placebo group residuals. Residuals vs Fitted values for the fixed effect model for the placebo group of individuals

FIG. 5. Data residuals. This is a model of only the fixed effects. The blue curve is the least squares line and the red curve is the smoothed loess fit.

FIG. 6. Q-Q plot, of placebo data. This is the QQ plot displaying the normality of the data for the model built in the model building section. This is for the placebo group only.

FIG. 7. QQ plot, of treatment arm data. This QQ plot is for the treatment only group using the model from the model building section. Normality is demonstrated, given that the data mostly follows the line y=x.

FIG. 8. Residuals of the model: placebo. This graph displays the residuals of the model in the model building section. No pattern in these residuals further supports the normality of the data.

FIG. 9. Residuals of the model: treatment group. This is a plot of the residuals of the model for only the treatment group. Again, no distinct pattern further supports normality.

FIG. 10. Data residuals for the model. This models the residuals of only the fixed effects in the linear model in the model building section. The red curve is the loess curve fit line and the blue is the least squares line.

FIG. 11. Linearity. Loess curve of principle components (PC) phase data. The Loess curve is in red.

FIG. 12. Treatment group trajectories. Selection of individual trajectories of UPDRS over time of a subset of individuals on treatment

FIG. 13. Placebo group trajectories. Selection of individual trajectories of UPDRS over time of a subset of individuals on Placebo.

### DETAILED DESCRIPTION OF THE INVENTION

R(+)-N-propargyl-l-aminoindan (“R-PAI”), also known as rasagiline, is a small molecule having the following chemical structure:

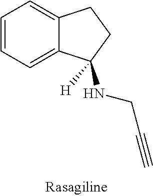

Rasagiline has been reported to be a selective inhibitor of the B-form of the enzyme monoamine oxidase (“MAO-B”) and is useful in treating Parkinson's disease and various other conditions by inhibition of MAO-B in the brain.

A pharmaceutically acceptable salt of rasagiline, rasagiline citrate, and the process of preparing the same has been described in U.S. Pat. No. 7,855,233, the entire content of which is hereby incorporated by reference.

Crystalline rasagiline, and the process of preparing the same has been described in U.S. Pat. Nos. 7,750,051, 7,968,749, the entire contents of which are hereby incorporated by reference.

Delayed release rasagiline formulations have been described in United States Application Publication Nos. 2009/0181086, 2010/0189790, 2010/0189788, 2010/0189787, and 2010/0189791, the entire content of each of which is hereby incorporated by reference.

This invention provides a method for treating a human subject afflicted with Parkinson's disease (PD) with a pharmaceutical composition comprising rasagiline or a pharmaceutically acceptable salt of rasagiline, and a pharmaceutically acceptable carrier, comprising the steps of:
(i) obtaining a biological sample comprising a genome from the human subject afflicted with Parkinson's disease;
(ii) assaying the DNA or RNA of the biological sample

from the human subject using probes or primers, to determine the diploid genotype of the human subject at single nucleotide polymorphism (SNP) rs1076560 or rs2283265;
(iii) identifying the human subject as a predicted responder to rasagiline if the diploid genotype is CC at rs1076560, CC at rs2283265, or CC at both rs1076560 and rs2283265; and
(iv) administering the pharmaceutical composition comprising rasagiline and a pharmaceutically acceptable carrier to the human subject if the human subject is identified as a predicted responder to rasagiline.

In one embodiment, step ii) further comprises assaying to determine the diploid genotype of the human subject at rs36023.

In one embodiment, further comprising identifying the human subject as a responder to rasagiline if the diploid genotype is AA at rs36023.

In one embodiment, the human subject is female.

In one embodiment, the human subject is male.

In one embodiment, the human subject is self-reported Caucasian.

In one embodiment, the human subject is self-reported non-Caucasian.

In one embodiment, the pharmaceutically acceptable salt is a tartrate, esylate, mesylate, or sulfate salt.

In one embodiment, the pharmaceutically acceptable salt is a mesylate salt.

In one embodiment, the pharmaceutical composition is a solid dosage form.

In one embodiment, the pharmaceutical composition is an oral dosage form.

In one embodiment, the pharmaceutical composition is in tablet form.

In one embodiment, the pharmaceutical composition comprises a 0.5-20.0 mg dose of rasagiline.

In one embodiment, the pharmaceutical composition comprises a 0.5-10.0 mg dose of rasagiline.

In one embodiment, the pharmaceutical composition comprises a 0.5-2.0 mg dose of rasagiline.

In one embodiment, the pharmaceutical composition comprises a 2.0 mg dose of rasagiline.

In one embodiment, the pharmaceutical composition comprises a 1.0 mg dose of rasagiline.

In one embodiment, the pharmaceutical composition comprises a 0.5 mg dose of rasagiline.

In one embodiment, the pharmaceutical composition comprising rasagiline and a pharmaceutically acceptable carrier is administered as monotherapy.

In one embodiment, the pharmaceutical composition comprising rasagiline and a pharmaceutically acceptable carrier is administered in combination at least one other Parkinson's disease drug.

In one embodiment, the method comprises determining the genotype of the subject at 2 or more of said SNPs.

In one embodiment, step iv) further comprises administering a pharmaceutical composition which does not comprise rasagiline to the subject if the subject is not a predicted responder.

In one embodiment, in the human subject is administered a pharmaceutical composition comprising bromocriptine, benztropine, levodopa, ropinirole, pramipexole, rotigotine, cabergoline, entacapone, tolcapone, amantadine or selegiline and a pharmaceutically acceptable carrier if the subject is not identified as a responder.

This invention also provides a method for treating a human subject afflicted with Parkinson's disease comprising the steps of:
(i) administering to the human subject a therapeutic amount of a pharmaceutical composition comprising rasagiline, or a pharmaceutically acceptable salt of rasagiline, and a pharmaceutically acceptable carrier;
(ii) obtaining a biological sample comprising a genome from the human subject afflicted with Parkinson's disease;
(iii) assaying the DNA or RNA of the biological sample from the human subject using probes or primers, to determine the diploid genotype of the human subject at single nucleotide polymorphism (SNP) rs1076560 or rs2283265;
(iv) identifying the human subject as a predicted responder to rasagiline if the diploid genotype is CC at rs1076560, CC at rs2283265, or CC at both rs1076560 and rs2283265; and
(v) continuing administration of the pharmaceutical composition if the human subject is identified as a predicted responder to rasagiline, or modifying the administration of the pharmaceutical composition to the human subject if the human subject is not identified as a predicted responder to rasagiline.

In one embodiment, step iii) further comprises assaying to determine the diploid genotype of the human subject at rs36023.

In one embodiment, further comprising identifying the human subject as a responder to rasagiline if the diploid genotype is AA at rs36023.

In one embodiment, step ii) is conducted 12, 24, or 36 weeks after initiation of administration of rasagiline or a pharmaceutically acceptable salt of rasagiline.

In one embodiment, step ii) is conducted 12 weeks after initiation of administration of rasagiline or a pharmaceutically acceptable salt of rasagiline.

In one embodiment, a predicted responder's rate of improvement of Parkinson's disease is quantified by the Total UPDRS score, wherein a sustained improvement is a reduction in UPDRS score of 3.5 or more than is first observed at either 12 or 24 weeks and persisted at 24 or 36 weeks, respectively.

In one embodiment, the method further comprises identifying the human subject as a predicted responder to rasagiline for a period of more than 12 weeks, more than 24 weeks, or more than 36 weeks.

In one embodiment, the pharmaceutically acceptable salt is a tartrate, esylate, mesylate, or sulfate salt.

In one embodiment, the pharmaceutically acceptable salt is a mesylate salt.

In one embodiment, the pharmaceutical composition is a solid dosage form.

In one embodiment, the pharmaceutical composition is an oral dosage form.

In one embodiment, the pharmaceutical composition is in tablet form.

In one embodiment, the pharmaceutical composition comprises a 0.5-20.0 mg dose of rasagiline.

In one embodiment, the pharmaceutical composition comprises a 0.5-10.0 mg dose of rasagiline.

In one embodiment, the pharmaceutical composition comprises a 0.5-2.0 mg dose of rasagiline.

In one embodiment, the pharmaceutical composition comprises a 2.0 mg dose of rasagiline.

In one embodiment, the pharmaceutical composition comprises a 1.0 mg dose of rasagiline.

In one embodiment, the pharmaceutical composition comprises a 0.5 mg dose of rasagiline.

In one embodiment, the genotype is determined from a nucleic acid-containing sample that has been obtained from the subject.

In one embodiment, the genotype is determined us restriction fragment length polymorphism (RFLP) analysis, sequencing, single strand conformation polymorphism analysis (SSCP), chemical cleavage of mismatch (CCM), denaturing high performance liquid chromatography (DHPLC), Polymerase Chain Reaction (PCR) or an array, or a combination thereof.

In one embodiment, the genotype is determined using at least one pair of PCR primers and at least one probe.

In one embodiment, the genotype is determined using an array. In one embodiment, the array is a gene array, DNA array, a DNA microarray, or a bead array.

In one embodiment, determining the genotype of the subject at said one or more SNPs comprises:
(i) obtaining DNA from a sample that has been obtained from the subject;
(ii) optionally amplifying the DNA; and
(iii) subjecting the DNA or the amplified DNA to restriction fragment length polymorphism (RFLP) analysis, sequencing, single strand conformation polymorphism analysis (SSCP), chemical cleavage of mismatch (CCM), gene chip, denaturing high performance liquid chromatography (DHPLC) and polymerase chain reaction (PCR), an array, or a combination thereof.

In one embodiment, the human subject is a naive patient.

In one embodiment, the human subject has been previously administered a Parkinson's disease drug other than rasagiline.

In one embodiment, the genotype of the subject at said one or more SNPs is obtained indirectly by determining the genotype of the subject at a SNP that is in linkage disequilibrium with said one or more SNPs.

In one embodiment, step ii) further comprises assaying to determine the diploid genotype of the human subject at rs36023 or rs1079597.

In one embodiment, the method further comprises identifying the human subject as a responder to rasagiline if the diploid genotype is AA at rs36023 and/or CC at rs1079597.

This invention also provides a diagnostic kit for evaluating responsiveness to treatment with rasagiline in a human subject afflicted with Parkinson's disease, the kit comprising
(i) at least one probe specific for SNP rs1076560 or rs2283265, and
(ii) instructions for use of the at least one probe to evaluate responsiveness of the subject to treatment with rasagiline.

In one embodiment, the kit further comprises
(i) at least one probe specific for SNP rs36023 or rs1079597, and
(ii) instructions for use of the at least one probe to evaluate responsiveness of the subject to treatment with rasagiline.

This invention also provides a diagnostic kit for evaluating responsiveness to treatment with rasagiline in a human subject afflicted with Parkinson's disease, the kit comprising
(i) at least one pair of PCR primers designed to amplify one or more DNA segments which include SNP rs1076560 or rs2283265, and
(ii) instructions for use of the at least one pair of PCR primers to evaluate responsiveness of the subject to treatment with rasagiline.

In one embodiment, the kit further comprises
(i) at least one pair of PCR primers designed to amplify one or more DNA segments which include SNP rs36023 or rs1079597, and
(ii) ceutical composition comprises a 1.0 mg dose of primers to evaluate responsiveness of the subject to treatment with rasagiline.

This invention also provides a PCR amplification kit comprising
(i) at least one pair of PCR primers designed to amplify one or more DNA segments which include SNP rs1076560 or rs2283265, and
(ii) instructions for use of the PCR primers to amplify the one or more segments of DNA.

In one embodiment, the PCR amplification kit further comprises
(i) at least one pair of PCR primers designed to amplify one or more DNA segments which include SNP rs36023 or rs1079597, and
(ii) instructions for use of the PCR primers to amplify the one or more segments of DNA.

This invention also provides a diagnostic kit for evaluating responsiveness to treatment with rasagiline in a human subject afflicted with Parkinson's disease, the kit comprising
(i) a reagent for performing restriction fragment length polymorphism (RFLP) analysis, sequencing, single strand conformation polymorphism analysis (SSCP), chemical cleavage of mismatch (CCM), gene chip, denaturing high performance liquid chromatography (DHPLC) and polymerase chain reaction (PCR) amplification for determining the identity of one or more SNPs wherein the one or more SNPs comprises at least one of rs1076560 or rs2283265, and
(ii) instructions for use of the reagent to evaluate responsiveness of the subject to treatment with rasagiline.

In one embodiment, the diagnostic kit further comprises
(i) a reagent for performing restriction fragment length polymorphism (RFLP) analysis, sequencing, single strand conformation polymorphism analysis (SSCP), chemical cleavage of mismatch (CCM), gene chip, denaturing high performance liquid chromatography (DHPLC) and polymerase chain reaction (PCR) amplification for determining the identity of one or more SNPs wherein the one or more SNPs comprises at least one of rs36023 or rs1079597, and
(ii) instructions for use of the reagent to evaluate responsiveness of the subject to treatment with rasagiline.

This invention also provides a method of determining the identity of the alleles of fewer than 10000 single nucleotide polymorphisms (SNPs) in a subject selected from the group of subjects consisting of human subjects diagnosed with Parkinson's disease to produce a polymorphic profile of the selected subject diagnosed with Parkinson's disease, comprising
(i) obtaining a biological sample comprising a genome from the selected subject diagnosed with Parkinson's disease;
(ii) selecting for allelic identity analysis at least a SNP located at rs1076560 and a SNP located at rs2283265 within the genome of the selected subject diagnosed with Parkinson's disease; and
(iii) assaying, with probes or primers, whether
   a) the allelic identity at rs1076560 is CC within the nucleotide sequence of the genome in the biological sample of step i), and
   b) the allelic identity at rs2283265 is CC within the nucleotide sequence of the genome in the biological sample of step i), and
wherein fewer than 10000 SNPs are selected for allelic identity analysis in step ii) and the same fewer than 10000 SNPs are assayed in step iii).

In one embodiment, the method further comprises
(i) obtaining a biological sample comprising a genome from the selected subject diagnosed with Parkinson's disease;
(ii) selecting for allelic identity analysis at least a SNP located at rs36023 and a SNP located at rs1079597 within the genome of the selected subject diagnosed with Parkinson's disease; and
(iii) assaying, with probes or primers, whether
   a) the allelic identity at rs36023 is AA within the nucleotide sequence of the genome in the biological sample of step i), and
   b) the allelic identity at rs1079597 is CC within the nucleotide sequence of the genome in the biological sample of step i), and
wherein fewer than 10000 SNPs are selected for allelic identity analysis in step ii) and the same fewer than 10000 SNPs are assayed in step iii).

In one embodiment, the method further comprises identifying the human subject as a predicted responder to rasagiline if the diploid genotype is CC at rs1079597, AA at rs36023, or CC at rs1079597 and AA at rs36023.

In one embodiment, the method further comprises directing the human subject to receive administration of a pharmaceutical composition comprising rasagiline and a pharmaceutically acceptable carrier.

In one embodiment, the method further comprises identifying the human subject as a predicted responder to rasagiline for a period of more than 12 weeks, more than 24 weeks, or more than 36 weeks.

In one embodiment,
if assaying the DNA or RNA of the biological sample from the human subject comprises a primer, then the assaying comprises
   i) hybridizing a primer to a nucleic acid having the sequence of a region proximal to the SNP located at rs1076560 or rs2283265; or
   ii) hybridizing a primer to a nucleic acid having the sequence of the SNP located at rs1076560 or rs2283265, and
if assaying the DNA or RNA of the biological sample from the human subject comprises a probe, then the assaying comprises
   i) hybridizing a probe to a nucleic acid having the sequence of the C allele located at rs1076560; or
   ii) hybridizing a probe to a nucleic acid having the sequence of the C allele located at rs2283265;

In one embodiment
if assaying the DNA or RNA of the biological sample from the human subject comprises a primer, then the assaying further comprises
   i) hybridizing a primer to a nucleic acid having the sequence of a region proximal to the SNP located at rs36023 or rs1079597; or
   ii) hybridizing a primer to a nucleic acid having the sequence of the SNP located at rs36023 or rs1079597, and
if assaying the DNA or RNA of the biological sample from the human subject comprises a probe, then the assaying further comprises
   i) hybridizing a probe to a nucleic acid having the sequence of the C allele located at rs1079597; or
   ii) hybridizing a probe to a nucleic acid having the sequence of the A allele located at rs36023;

In one embodiment the method further comprises producing a polymorphic profile of the human subject based on the identity of the alleles assayed.

In one embodiment the polymorphic profile is on a physical or electronic report, and the physical or electronic report identifies whether the human subject is a predicted responder to rasagiline based on the polymorphic profile.

In one embodiment, assaying the DNA or RNA of the biological sample from the human subject using is with a probe, and the probe is on an array.

In one embodiment, the array comprises at least one probe that is fully complementary to
i) a nucleic acid having the sequence of the C allele located at rs1076560; or
ii) a nucleic acid having the sequence of the C allele located at rs2283265;

In one embodiment the array further comprises at least one probe that is fully complementary to
i) a nucleic acid having the sequence of the C allele located at rs1079597; or
ii) a nucleic acid having the sequence of the A allele located at rs36023.

This invention also provides a physical or electronic database comprising the polymorphic profiles of human subjects afflicted with PD, wherein each polymorphic profile includes the diploid genotype of fewer than 10000 SNPs, and the fewer than 10000 SNPs include rs1076560, and rs36023.

In one embodiment, the fewer than 10000 SNPs further include rs36023 or rs1079597.

As used herein, a genetic marker refers to a DNA sequence that has a known location on a chromosome and displays variability between individuals in its nucleotide carriage status. Several non-limiting examples of classes of genetic markers include SNP (single nucleotide polymorphism), STR (short tandem repeat), SFP (single feature polymorphism), VNTR (variable number tandem repeat), microsatellite polymorphism, insertions and deletions. The genetic markers associated with the invention are SNPs. As used herein a SNP or “single nucleotide polymorphism” refers to a specific site in the genome where there is a difference in DNA base (i.e. nucleotide) between individuals. In some embodiments the SNP is located in a coding region of a gene. In other embodiments the SNP is located in a noncoding region of a gene. In still other embodiments the SNP is located in an intergenic region.

Several non-limiting examples of databases from which information on SNPs or genes that are associated with human disease can be retrieved include: NCBI resources, The SNP Consortium LTD, NCBI dbSNP database, International HapMap Project, 1000 Genomes Project, Glovar Variation Browser, SNPStats, PharmGKB, GEN-SniP, and SNPedia.

In some embodiments, SNPs associated with the invention comprise one or more of the SNPs listed in Tables 7-9. In some embodiments, multiple SNPs are evaluated simultaneously while in other embodiments SNPS are evaluated separately. SNPs are identified herein using the rs identifier numbers in accordance with the NCBI dbSNP database, which is publically available at: http://www.ncbi.nlm.nih.gov/projects/SNP/.

In some embodiments, SNPs in linkage disequilibrium with the SNPs associated with the invention are useful for obtaining similar results. As used herein, linkage disequilibrium refers to the non-random association of SNPs at one locus. Techniques for the measurement of linkage disequilibrium are known in the art. As two SNPs are in linkage disequilibrium if they are inherited together more often than randomly selected, the information they provide is correlated to a certain extent. SNPs in linkage disequilibrium with the SNPs included in the models can be obtained from databases such as HapMap or other related databases, from experimental setups run in laboratories or from computer-aided in-silico experiments. Determining the genotype of a subject at a position of SNP as specified herein, e.g. as specified by NCBI dbSNP rs identifier, may comprise directly genotyping, e.g. by determining the identity of the nucleotide of each allele at the locus of SNP, and/or indirectly genotyping, e.g. by determining the identity of each allele at one or more loci that are in linkage disequilibrium with the SNP in question and which allow one to infer the identity of each allele at the locus of SNP in question with a substantial degree of confidence (sometimes referred to as imputation). In some cases, indirect genotyping may comprise determining the identity of each allele at one or more loci that are in sufficiently high linkage disequilibrium with the SNP in question so as to allow one to infer the identity of each allele at the locus of SNP in question with a probability of at least 85%, at least 90% or at least 99% certainty.

An allele at a position of SNP (allele “at a” SNP) may be represented by a single letter which corresponds to the identity of one of the two nucleotides that an individual carries at the SNP, given that an individual carries two chromosomes across the genome (i.e., one inherited from their biological mother and one from their biological father), where A represents adenine, T represents thymine, C represents cytosine, and G represents guanine. The identity of two alleles at a single SNP which comprises the genotype (i.e. both nucleotides carried by the individual at the SNP) may be represented by a two letter combination of A, T, C, and G, where the first letter of the two letter combination represents one allele and the second letter represents the second allele, and where A represents adenine, T represents thymine, C represents cytosine, and G represents guanine. Thus, a two allele genotype at a SNP can be represented as, for example, AA, AT, AG, AC, TT, TG, TC, GG, GC, or CC. It is understood that AT, AG, AC, TG, TC, and GC are equivalent to TA, GA, CA, GT, CT, and CG, respectively.

The SNPs of the invention can be used as predictive indicators of the response to rasagiline in subjects afflicted with Parkinson's disease. Aspects of the invention relate to determining the presence of SNPs through obtaining a patient DNA sample and evaluating the patient sample for the genotype carried at one or more SNPs, or for a certain set of SNPs. It should be appreciated that a patient DNA sample can be extracted, and a SNP can be detected in the sample, through any means known to one of ordinary skill in art. Some non-limiting examples of known techniques include detection via restriction fragment length polymorphism (RFLP) analysis, microarrays including but not limited to planar microarrays or bead arrays, sequencing, single strand conformation polymorphism analysis (SSCP), chemical cleavage of mismatch (CCM), and denaturing high performance liquid chromatography (DHPLC). In some embodiments, a SNP is detected through PCR amplification and sequencing of the DNA region comprising the SNP. In some embodiments SNPs are detected using DNA microarrays, also called DNA Chips. Microarrays for detection of genetic polymorphisms, changes or mutations (in general, genetic variations) such as a SNP in a DNA sequence, comprise a solid surface, typically glass, on which a high number of genetic sequences are deposited (the probes), complementary to the genetic variations to be studied. Using standard robotic printers to apply probes to the array a high density of individual probe features can be obtained, for example probe densities of 600 features per cm² or more can be typically achieved. The positioning of probes on an array is precisely controlled by the printing device (robot, inkjet printer, photolithographic mask etc) and probes are aligned in a grid. The organization of probes on the array facilitates the subsequent identification of specific probe-target interactions. Additionally it is common, but not necessary, to divide the array features into smaller sectors, also grid-shaped, that are subsequently referred to as sub-arrays. Sub-arrays typically comprise 32 individual probe features although lower (e.g. 16) or higher (e.g. 64 or more) features can comprise each subarray. In some embodiments, detection of genetic variation such as the presence of a SNP involves hybridization to sequences which specifically recognize the normal and the mutant allele in a fragment of DNA derived from a test sample. Typically, the fragment has been amplified, e.g. by using the polymerase chain reaction (PCR), and labelled e.g. with a fluorescent molecule. A laser can be used to detect bound labelled fragments on the chip and thus an individual who is homozygous for the normal allele can be specifically distinguished from heterozygous individuals (in the case of autosomal dominant conditions then these individuals are referred to as carriers) or those who are homozygous for the mutant allele. In some embodiments, the amplification reaction and/or extension reaction is carried out on the microarray or bead itself. For differential hybridization based methods there are a number of methods for analyzing hybridization data for genotyping: Increase in hybridization level: The hybridization levels of probes complementary to the normal and mutant alleles are compared. Decrease in hybridization level: Differences in the sequence between a control sample and a test sample can be identified by a decrease in the hybridization level of the totally complementary oligonucleotides with a reference sequence. A loss approximating 100% is produced in mutant homozygous individuals while there is only an approximately 50% loss in heterozygotes. In Microarrays for examining all the bases of a sequence of “n” nucleotides (“oligonucleotide”) of length in both strands, a minimum of “2n” oligonucleotides that overlap with the previous oligonucleotide in all the sequence except in the nucleotide are necessary. Typically the size of the oligonucleotides is about 25 nucleotides. However it should be appreciated that the oligonucleotide can be any length that is appropriate as would be understood by one of ordinary skill in the art. The increased number of oligonucleotides used to reconstruct the sequence reduces errors derived from fluctuation of the hybridization level. However, the exact change in sequence cannot be identified with this method; in some embodiments this method is combined with sequencing to identify the mutation. Where amplification or extension is carried out on the microarray or bead itself, three methods are presented by way of example: In the Minisequencing strategy, a mutation specific primer is fixed on the slide and after an extension reaction with fluorescent dideoxynucleotides, the image of the Microarray is captured with a scanner. In the Primer extension strategy, two oligonucleotides are designed for detection of the wild type and mutant sequences respectively. The extension reaction is subsequently carried out with one fluorescently labelled nucleotide and the remaining nucleotides unlabelled. In either case the starting material can be either an RNA sample or a DNA product amplified by PCR. In the Tag arrays strategy, an extension reaction is carried out in solution with specific primers, which carry a determined 5¹ sequence or “tag”. The use of Microarrays with oligonucleotides complementary to these sequences or “tags” allows the capture of the resultant products of the extension. Examples of this include the high density Microarray “Flex-flex” (Affymetrix). In the Illumina 1M Dou BeadChip array (http://www.illumina.com/products/humanlm_duo_dna_analysis_beadchip kits.ilmn), SNP genotypes are generated from fluorescent intensities using the manufacturer's default cluster settings.

Also within the scope of the invention are kits and instructions for their use. In some embodiments kits associated with the invention are kits for identifying one or more SNPs within a patient sample. In some embodiments a kit may contain primers for amplifying a specific genetic locus. In some embodiments, a kit may contain a probe for hybridizing to a specific SNP. The kit of the invention can include reagents for conducting each of the following assays including but not limited to restriction fragment length polymorphism (RFLP) analysis, microarrays including but not limited to planar microarrays or bead arrays, sequencing, single strand conformation polymorphism analysis (SSCP), chemical cleavage of mismatch (CCM), and denaturing high performance liquid chromatography (DHPLC), PCR amplification and sequencing of the DNA region comprising the SNP. A kit of the invention can include a description of use of the contents of the kit for participation in any biological or chemical mechanism disclosed herein. A kit can include instructions for use of the kit components alone or in combination with other methods or compositions for assisting in screening or diagnosing a sample and/or determining whether a subject is a responder or a non-responder to rasagiline.

Every embodiment disclosed herein can be combined with every other embodiment of the subject invention, unless specified otherwise.

The preferred dosages of R(+)PAI in any of the disclosed compositions may be within the following ranges: for oral or suppository formulations 0.01-20 mg per dosage unit to be taken daily, preferably 0.5-5 mg per dosage unit to be taken daily and more preferably 1 mg or 2 mg per dosage unit to be taken daily may be used.

By any range disclosed herein, it is meant that all hundredth, tenth and integer unit amounts within the range are specifically disclosed as part of the invention. Thus, for example, 0.01 mg to 50 mg means that 0.02, 0.03 . . . 0.09; 0.1, 0.2 . . . 0.9; and 1, 2 . . . 49 mg unit amounts are included as embodiments of this invention.

It will be noted that the structure of the compound of this invention includes an asymmetric carbon atom and thus the compound occurs as racemate, racemic mixture, and isolated single enantiomers. All such isomeric forms of these compounds are expressly included in this invention. Each stereogenic carbon may be of the R or S configuration. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis, such as those described in “Enantiomers, Racemates and Resolutions” by J. Jacques, A. Collet and S. Wilen, Pub. John Wiley & Sons, NY, 1981. For example, the resolution may be carried out by preparative chromatography on a chiral column.

The subject invention is also intended to include all isotopes of atoms occurring on the compounds disclosed herein. Isotopes include those atoms having the same atomic number but different mass numbers. By way of general example and without limitation, isotopes of hydrogen include tritium and deuterium. Isotopes of carbon include C-13 and C-14.

It will be noted that any notation of a carbon in structures throughout this application, when used without further notation, are intended to represent all isotopes of carbon, such as ¹²C, ¹³C, or ¹⁴C. Furthermore, any compounds containing ¹³C or ¹⁴C may specifically have the structure of any of the compounds disclosed herein.

It will also be noted that any notation of a hydrogen in structures throughout this application, when used without further notation, are intended to represent all isotopes of hydrogen, such as ¹H, ²H, or ³H. Furthermore, any compounds containing ²H or ³H may specifically have the structure of any of the compounds disclosed herein.

Isotopically-labeled compounds can generally be prepared by conventional techniques known to those skilled in the art or by processes analogous to those described in the Examples disclosed herein using an appropriate isotopically-labeled reagents in place of the non-labeled reagents employed.

A characteristic of a compound refers to any quality that a compound exhibits, e.g., peaks or retention times, as determined by 1H nuclear magnetic spectroscopy, mass spectroscopy, infrared, ultraviolet or fluorescence spectrophotometry, gas chromatography, thin layer chromatography, high performance liquid chromatography (HPLC), elemental analysis, Ames test, dissolution, stability and any other quality that can be determined by an analytical method. Once the characteristics of a compound are known, the information can be used to, for example, screen or test for the presence of the compound in a sample. Quantity or weight percentage of a compound present in a sample can be determined by a suitable apparatus, for example, a HPLC.

As used herein, a “pharmaceutically acceptable salt” of rasagiline includes citrate, tannate, malate, mesylate, maleate, fumarate, tartrate, esylate, p-toluenesulfonate, benzoate, acetate, phosphate and sulfate salts. For the preparation of pharmaceutically acceptable acid addition salts of the compounds of the invention, the free base can be reacted with the desired acids in the presence of a suitable solvent by conventional methods.

Rasagiline can also be used in its free base form. A process of manufacture of the rasagiline free base is described in U.S. Pat. Nos. 7,750,051 and 7,968,749, the contents of which are hereby incorporated by reference.

As used herein, “drug substance” refers to the active ingredient in a drug product, which provides pharmacological activity or other direct effect in the diagnosis, cure, mitigation, treatment, or prevention of disease, or to affect the structure or any function of the body of man or animals.

As used herein, “drug product” refers to the finished dosage form containing the drug substance as well as at least one pharmaceutically acceptable carrier.

As used herein, an “isolated” compound is a compound isolated from the crude reaction mixture following an affirmative act of isolation. The act of isolation necessarily involves separating the compound from the other known components of the crude reaction mixture, with some impurities, unknown side products and residual amounts of the other known components of the crude reaction mixture permitted to remain. Purification is an example of an affirmative act of isolation.

As used herein, a composition that is “free” of a chemical entity means that the composition contains, if at all, an amount of the chemical entity which cannot be avoided following an affirmative act intended to purify the composition by separating the chemical entity from the composition.

As used herein, “stability testing” refers to tests conducted at specific time intervals and various environmental conditions (e.g., temperature and humidity) to see if and to what extent a drug product degrades over its designated shelf life time. The specific conditions and time of the tests are such that they accelerate the conditions the drug product is expected to encounter over its shelf life. For example, detailed requirements of stability testing for finished pharmaceuticals are codified in 21 C.F.R §211.166, the entire content of which is hereby incorporated by reference.

As used herein, a pharmaceutical composition which is “X weeks old” refers to the period of time, in this case one week, since the pharmaceutical composition was made.

As used herein, “ambient temperature” refers a temperature of from about 20° C. to about 30° C.

A “detection limit” for an analytical method used in screening or testing for the presence of a compound in a sample is a threshold under which the compound in a sample cannot be detected by the analytical method, e.g. an HPLC, MS, NMR, or FT-IR method.

As used herein, “about” in the context of a measurable numerical value means the numerical value within the standard error of the analytical method used to measure.

A dosage unit may comprise a single compound or mixtures of compounds thereof. A dosage unit can be prepared for oral dosage forms, such as tablets, capsules, pills, powders, and granules.

As used herein, a “pharmaceutically acceptable” carrier or excipient is one that is suitable for use with humans and/or animals without undue adverse side effects (such as toxicity, irritation, and allergic response) commensurate with a reasonable benefit/risk ratio.

Specific examples of pharmaceutical acceptable carriers and excipients that may be used to formulate oral dosage forms are described, e.g., in U.S. Pat. No. 6,126,968 to Peskin et al., issued Oct. 3, 2000. Techniques and compositions for making dosage forms useful in the present invention are described-in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

Tablets may contain suitable binders, lubricants, disintegrating agents, coloring agents, flavoring agents, flow-inducing agents, melting agents, stabilizing agents, solubilizing agents, antioxidants, buffering agent, chelating agents, fillers and plasticizers. For instance, for oral administration in the dosage unit form of a tablet or capsule, the active drug component can be combined with an oral, non-toxic, pharmaceutically acceptable, inert carrier such as gelatin, agar, starch, methyl cellulose, dicalcium phosphate, calcium sulfate, mannitol, sorbitol and the like. Suitable binders include starch, gelatin, natural sugars such as corn starch, natural and synthetic gums such as acacia, tragacanth, or sodium alginate, povidone, carboxymethylcellulose, polyethylene glycol, waxes, and the like. Antioxidants include ascorbic acid, fumaric acid, citric acid, malic acid, gallic acid and its salts and esters, butylated hydroxyanisole, editic acid. Lubricants used in these dosage forms include sodium oleate, sodium stearate, sodium benzoate, sodium acetate, stearic acid, sodium stearyl fumarate, talc and the like. Disintegrators include, without limitation, starch, methyl cellulose, agar, bentonite, xanthan gum, croscarmellose sodium, sodium starch glycolate and the like, suitable plasticizers include triacetin, triethyl citrate, dibutyl sebacate, polyethylene glycol and the like.

The Total UPDRS (Unified Parkinson's Disease Rating Scale) score represents the level or severity of Parkinson's disease symptoms. It is used for measuring the change from baseline in efficacy variables during the treatment. UPDRS consists of a three-part test. A total of 31 items are included in Parts I, II and III test. Each item receives a score ranging from 0 to 4 where 0 represents the absence of impairment and 4 represents the highest degree of impairment. The sum of Parts I, II and III at each study visit provides a Total UPDRS score. Part I is designed to rate mentation, behavior and mood (items 1-4). It is collected as historical information. Part II (items 5-17) is also historical information. Part III (items 18-31) is a motor examination at the time of a visit.

### Part I: Mentation, Behavior and Mood

### Item 1. Intellectual Impairment

0: None.
1: Mild—Consistent forgetfulness with partial recollection of events and no other difficulties.
2: Moderate memory loss, with disorientation and moderate difficulty in handling complex problems. Mild but definitive impairment of function at home with need of occasional prompting.
3: Severe memory loss with disorientation for time and often to place. Severe impairment in handling problems.
4: Severe memory loss with orientation preserved to person only. Unable to make judgments or solve problems. Requires much help with personal care; cannot be left alone at all.

### Item 2. Thought Disorders (Due to Dementia or Drug Intoxication)

0: None.
1: Vivid dreaming.
2: Benign hallucinations with insight retained.
3: Occasional to frequent hallucinations or delusions; without insight; could interfere with daily activities.
4: Persistent hallucinations, delusions or florid psychosis. Not able to care for self.

### Item 3. Depression

0: Not present.
1: Periods of sadness or guilt greater than normal. Never sustained for days or weeks.
2: Sustained depression (1 week or more)
3: Sustained depression with vegetative symptoms (insomnia, anorexia, weight loss, loss of interest).
4: Sustained depression with vegetative symptoms and suicidal, thoughts or intent.

### Item 4. Motivation/Initiative

0: Normal.
1: Less assertive than usual; more passive.
2: Loss of initiative or disinterest in elective (non-routine) activities.
3: Loss of initiative or disinterest in day-to-day (routine) activities.
4: Withdrawn, complete loss of motivation.

### Part II: Activities of Daily Living (Score 0-4)

### Item 5. Speech

0: Normal.
1: Mildly affected. No difficulty being understood.
2: Moderately affected. Sometimes asked to repeat statements.
3: Severely affected. Frequently asked to repeat statements.
4: Unintelligible most of the time.

### Item 6. Salivation

0: Normal.
1: Slight but definite excess of saliva in mouth; may have nighttime drooling.
2: Moderately excessive of saliva; may have minimal drooling.
3: Marked excess of saliva with some drooling.
4: Marked drooling, requires constant tissue or handkerchief.

### Item 7. Swallowing

0: Normal.
1: Rare choking.
2: Occasional choking.
3: Requires soft food.
4: Requires nasogastric tube or gastrotomy feeding.

### Item 8. Handwriting

0: Normal.
1: Slightly slow or small.
2: Moderately slow or small; all words are legible.
3: Severely affected; not all words are legible.
4: The majority of words are not legible.

### Item 9. Cutting Food, Handling Utensils

0: Normal.
1: Somewhat slow, but no help needed.
2: Can cut most foods, although clumsy and slow; some help needed.
3: Food must be cut by someone, but can still feed slowly.
4: Needs to be fed.

### Item 10. Dressing

0: Normal.
1: Somewhat slow, but no help needed.
2: Occasional assistance with buttoning, getting arms in sleeves.
3: Considerable help required, but can do some things alone.
4: Helpless.

### Item 11. Hygiene

0: Normal.
1: Somewhat slow, but no help needed.
2: Needs help to shower or bathe, or very slow in hygienic care.
3: Requires assistance for washing, brushing teeth, combing hair, going to bathroom.
4: Foley catheter or other mechanical aids.

### Item 12. Turning in Bed and Adjusting Bed Clothes

0: Normal.
1: Somewhat slow and clumsy, but no help needed.
2: Can turn alone or adjust sheets, but with great difficulty.
3: Can initiate, but not turn or adjust sheets alone.
4: Helpless.

### Item 13. Falling (Unrelated to Freezing)

0: None.
1: Rare falling.
2: Occasionally falls, less than once per day.
3: Falls an average of once daily.
4: Falls more than once daily.
   Item 14. Freezing when Walking
0: None.
1: Rare freezing when walking; may have start-hesitation.
2: Occasional freezing when walking.
3: Frequent freezing. Occasionally falls from freezing.
4: Frequent falls from freezing.

### Item 15. Walking

0: Normal.
1: Mild difficulty. May not swing arms or may tend to drag leg.
2: Moderate difficulty, but requires little or no assistance.
3: Severe disturbance of walking, requiring assistance.
4: Cannot walk at all, even with assistance.

### Item 16. Tremor

0: Absent.
1: Slight and infrequently present.
2: Moderate; bothersome to patient.
3: Severe; interferes with many activities.
4: Marked; interferes with most activities.

### Item 17. Sensory Complaints Related to Parkinsonism

0: None.
1: Occasionally has numbness, tingling or mild aching.
2: Frequently has numbness, tingling or aching; not distressing.
3: Frequent painful sensations.
4: Excruciating pain.

### Part III: Motor Examination (Score 0-4)

### Item 18. Speech

0: Normal.
1: Slight loss of expression, diction and/or volume.
2: Monotone, slurred but understandable; moderately impaired.
3: Marked impairment, difficult to understand.
4: Unintelligible.

### Item 19. Facial Expression

0: Normal.
1: Minimal hypomimia, could be normal “Poker Face”.
2: Slight but definitely abnormal diminution of facial expression.
3: Moderate hypomimia; lips parted some of the time.
4: Masked or fixed faces with severe or complete loss of facial expression; lips parted ¼ inch or more.

### Item 20. Tremor at Rest

a) Face, lips and chin
b) Right hand
c) Left hand
d) Right foot
e) Left foot
0: Absent.
1: Slight and infrequently present.
2: Mild in amplitude and persistent; or moderate in amplitude, but only intermittently present.
3: Moderate in amplitude and present most of the time.
4: Marked in amplitude and present most of the time.

### Item 21. Action or Postural Tremor of Hands

0: Absent.
1: Slight; present with action.
2: Moderate in amplitude, present with action.
3: Moderate in amplitude with posture holding as well as action.
4: Marked in amplitude; interfere with feeding.
   Item 22. Rigidity (Judged on Passive Movement of Major Joints with Subject Relaxed In Sitting Position. Cogwheeling to be Ignored)
a) neck
b) right upper extremities
c) left upper extremities
d) right lower extremities
e) left lower extremities
0: Absent.
1: Slight or detectable only when activated by mirror or other movements.
2: Mild or moderate.
3: Marked, but full range of motion easily achieved.
4: Severe, range of motion achieved with difficulty.
   Item 23. Finger Taps (Subject Taps Thumb with Index Finger in Rapid Succession with Widest Amplitude Possible, Each Hand Separately)
a) Right hand
b) Left hand
0: Normal >15/5 sec.
1: Mild slowing and/or reduction in amplitude (11-14.5 sec).
2: Moderately impaired. Definite and early fatiguing. May have occasional arrests in movement (7-10/5 sec).
3: Severely impaired. Frequent hesitation in initiating movements or arrests in ongoing movement (3-6/5 sec).
4: Can barely perform the task (0-2/5 sec).
   Item 24. Hand Movement (Subject Opens and Closes Hands in Rapid Succession with Widest Amplitude Possible, Each Hand Separately)
a) Right hand
b) Left hand
0: Normal.
1: Mild slowing and/or reduction in amplitude.
2: Moderately impaired. Definite and early fatiguing. May have occasional arrests in movements.
3: Severely impaired. Frequent hesitation in initiating movements or arrests in ongoing movement.
4: Can barely perform the task.
   Item 25. Rapid Alternating Movements of Hands (Pronation, Supination Movements of Hands, Vertically or Horizontally with as Large an Amplitude as Possible, Both Hands Simultaneously)
0: Normal.
1: Mild slowing and/or reduction in amplitude.
2: Moderately impaired. Definite and early fatiguing. May have occasional arrests in movement.
3: Severely impaired. Frequent hesitation in initiating movements or arrests in ongoing movement.
4: Can barely perform the task.
   Item 26. Leg Agility (Subject Taps Heel on Ground in Rapid Succession, Picking Up Entire Leg. Amplitude should be about 3 Inches)
0: Normal.
1: Mild slowing and/or reduction in amplitude.
2: Moderately impaired. Definite and early fatiguing. May have occasional arrests in movement.
3: Severely impaired. Frequent hesitation in initiating movements or arrests in ongoing movement.
4: Can barely perform the task.
   Item 27. Arising from Chair (Subject Attempts to Arise from a Straight-Back Wood or Metal Chair with Arms Folded Across)
0: Normal.
1: Slow, or may need more than one attempt.
2: Pushes self up from arms of seat.
3: Tends to fall back and may have to try more than one time, but can get up without help.
4: Unable to arise without help.

### Item 28. Posture

0: Normal erect.
1: Not quite erect, slightly stooped posture; could be normal for older person.
2: Moderately stooped posture, definitely abnormal, can be

slightly leaning to one side.
3: Severely stooped posture with kyphosis; can be moderately leaning to one side.
4: Marked flexion with extreme abnormality of posture.

### Item 29. Gait

0: Normal.
1: Walks slowly, may shuffle with short steps, but no festination or propulsion.
2: Walks with difficulty, but requires little or no assistance; may have some festination, short steps, or propulsion.
3: Severe disturbance, of gait requiring assistance.
4: Cannot walk at all, even with assistance.

### Item 30. Postural Stability (Response to Sudden Posterior Displacement)

0: Normal.
1: Retropulsion, but recovers unaided.
2: Absence of postural response; would fall if not caught by examiner.
3: Very unstable, tends to lose balance spontaneously.
4: Unable to stand without assistance.

### Item 31. Body Bradykinesia and Hypokinesia (Combining Slowness, Hesitancy, Decreased Arm Swing, Small Amplitudes and Poverty of Movement in General)

0: None.
1: Minimal slowness, giving movement a deliberate character;

could be normal for some person. Possibly reduced amplitude.
2: Mild degree of slowness and poverty of movement which is definitely abnormal. Alternatively, some reduced amplitude.
3: Moderate slowness, poverty or small amplitude of movement.
4: Marked slowness, poverty or small amplitude of movement.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

### Methods

### Subjects

Men and women between the ages of 30 and 80 years old who had been diagnosed with Parkinson's disease (PD) within 18 months of a double-blind, delayed-start trial of rasagiline in Parkinson's disease (“ADAGIO”) [Olanow et al, 2009]trial start date, but who were not receiving treatment were recruited. The diagnosis was based on the UK Parkinson's Disease Society Brain Bank Criteria for clinically probable disease and included the presence of at least two of three features of the disease: resting tremor, bradykinesia, or rigidity. If resting tremor was not present, then unilateral onset of symptoms was required. In total, 1176 patients were recruited for the ADAGIO trial of which 805 provided consent for the pharmacogenetic sub-study. Seven hundred and fifty three samples were transferred to the Neurogenetics Laboratory at the Centre for Addiction and Mental Health (CAMH; Toronto, Canada).

Patients in the ADAGIO trial were recruited from 129 centers in 14 countries and were assigned to different treatment and dosage groups in a balanced fashion according to a centralized, computer-generated randomization schedule. The treatment groups were “delayed start” and “early start” of treatment with rasagiline. Within the two treatment groups, patients received either 1 mg or 2 mg of rasagiline. Patients were assessed at baseline and followed for 72 weeks with follow-up data available at approximately the 12, 24, 36, 42, 48, 54, 60, 66 and 72 week visits. For the first 36 weeks, individuals in the delayed start group received placebo and were then switched to active rasagiline treatment. The early start group received rasagiline following baseline assessment. Both treatment paradigms were coupled with matched placebo treatment arms. If PD symptoms progressed to the point of requiring anti-Parkinsonian therapy based on the investigator's assessment, individuals were started on appropriate therapy and withdrawn from the trial or they were allowed to switch to rasagiline treatment early. All response data from the delayed start group and the first 36 weeks of response data for the early start group, including their matched placebo groups (i.e., the delayed start group while on placebo), was available for the pharmacogenetic study.

### Single Nucleotide Polymorphism (SNP) Selection and Genotyping

A total of 204 SNPs and 5 Variable Number Tandem Repeats (VNTRs) from 28 candidate genes were genotyped. The genes were selected based on the role of the gene product in rasagiline's mode of action, metabolism, [Bar-Am et al. 2010; Chen and Swope 2005; Chen and Ly 2006] or association with PD susceptibility in previous genome wide association studies (GWAS) [2011; Do et al. 2011; Nalls et al. 2011]. The genes included dopamine receptors, catecholamine synthetic and catabolic enzymes, as well as catecholamine transporters, cytochrome P450 1A2 (CYP1A2), and glyceraldehyde-3-phosphate dehydrogenase (GAPDH) [Bar-Am, Weinreb, Amit, and Youdim 2010; Chen and Swope2005; Chen and Ly2006]. Functional SNPs as well as tag SNPs were selected for investigation; Tag SNP selection parameters: r²=0.8, mean allele frequency (MAF)=10%, coverage >80% per gene, covering 10 kb on either side of the translated region per gene. The SNPs were genotyped on the LifeTechnologies OpenArray NT genotyping platform (Grand Island, N.Y.). This platform allows for rapid analysis of up to 90,000 SNP genotypes per day. Briefly, the extracted DNA was mixed with reagents optimized for the OpenArray reaction. The combined mixture was robotically loaded onto an array containing a user-defined set of specific oligonucleotide primers and probes for the analysis of the SNPs of interest. The reaction then underwent standard polymerase chain reaction (PCR) amplification and the products were visualized and automatically genotyped using the QuantStudio Real-Time PCR system and software (Grand Island, N.Y.). The VNTRs [in the DAT1 VNTR, DRD4, LPR (including the rs25531 SNP), and MAOA genes] were amplified using standard PCR cycling methods and electrophoresed on the Applied Biosystems 3130 Genetic Analyzer (Grand Island, N.Y.). MAOA rs6323 was amplified using standard PCR cycling methods, digested overnight with the enzyme Fnu4HI and electrophoresed on an agarose gel. The sex-specific Amelogenin marker was used to determine the sex of each individual.

### Genetic Data Quality Control (QC)

After genotyping, there were a total of 753 samples with genetic data—including 19 intentional duplicates for QC. The investigators and laboratory technicians were blinded with respect to prior knowledge of duplicate sample status. In addition, a minimum of 10% of samples were genotyped in duplicate for every marker, and markers with a genotype call rate lower than 90% were re-genotyped. Samples were excluded based on non-Caucasian ancestry, duplicate status, low-genotyping rate, outlying heterozygosity, sex discrepancies and other agreed upon exclusions detailed below.

### Ancestry:

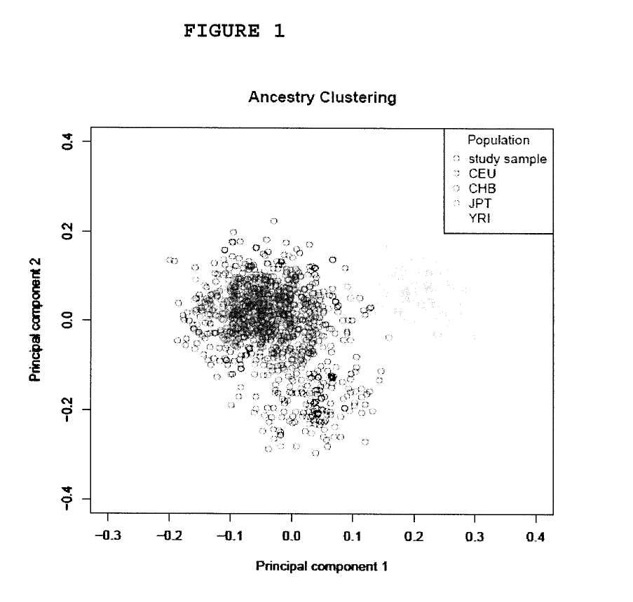

For the entire population, principal components analysis was performed using the SNP data to investigate the patterns between the principal components and ancestry. See, FIG. 1.

People clustered according to self reported ancestry and as there were too few markers to provide complete separation of the populations the former was used for exclusion of non-Caucasian individuals.

### Duplicate Samples:

Pairwise identity by descent (IBD) was calculated using the PLINK software [Harvard University, Massachusetts] and duplicate samples were removed (i.e., the duplicate with the lower genotyping call rate was excluded from analyses). A discussion of PLINK is provided at Purcell et al., 2007.

### Genotyping Rate:

Individuals with a missing genotype rate of more than 10% were removed.

### Heterozgosity:

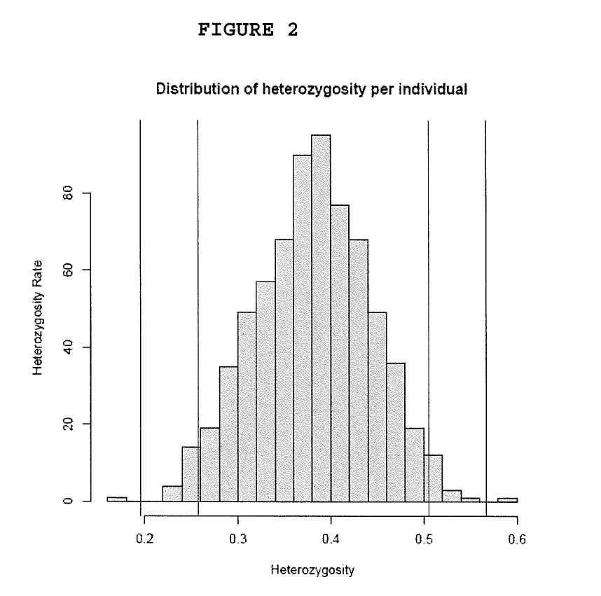

Overall marker heterozygosity was calculated for each sample and two individuals were removed as outliers. See, FIG. 2.

### Duplicate Genotyping:

The overall error rate from the duplicate genotyping was <1%. Of those markers retyped due to low genotype rate four individuals had greater than 10 genotype discrepancies, so these individuals were removed.

### Sex Discrepancies:

Four males who were heterozygous at the sex-linked SNPs were removed and 2 samples were removed for sex discrepancies.

### Other Discrepancies:

Duplicates identified by IBD analysis were removed to err on the side of caution. Once the quality control was completed, there were 694 individuals remaining for possible inclusion in the statistical analyses.

### Marker QC:

3 SNPs were removed for outlying Hardy Weinberg Equilibrium (HWE), p-value of <0.001: rs2069514, rs36024 and rs11868035. There were 2 SNPs with a low MAF, rs2069514 with MAF of 3.2% and rs2735917 with a MAF of 3.4%.

### Normality, Linearity, Independence and Equal Variance Assumptions

All figures and calculations in this section and the model building section were performed using the data from participants during the first 36 weeks of the study. This included data from the placebo phase of the delayed start group and the active rasagiline treatment of the early start group. Normality was determined.

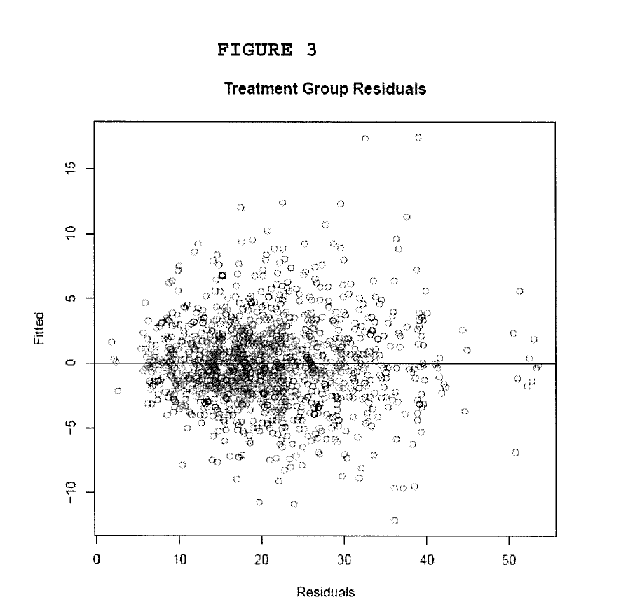

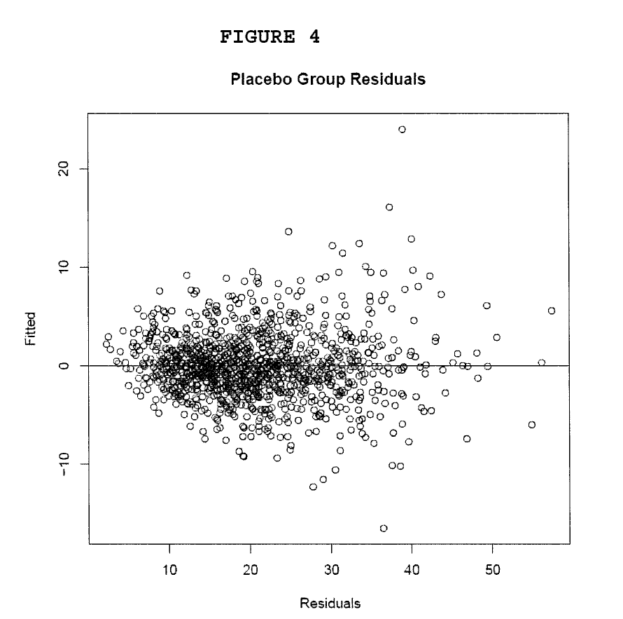

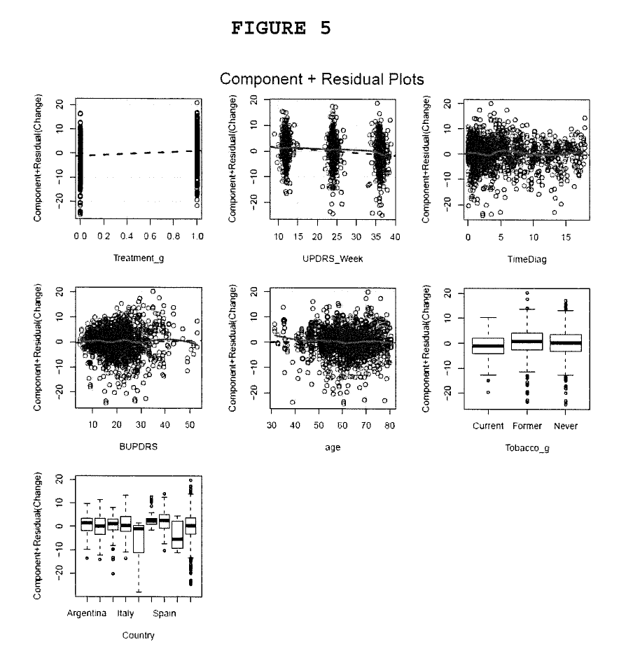

FIGS. 3-4 display the residuals of the model; there were no patterns visible in the data, which confirms equal variance. FIG. 5 demonstrates that there were no patterns in the data when the residuals were plotted against each covariate, demonstrating independence.

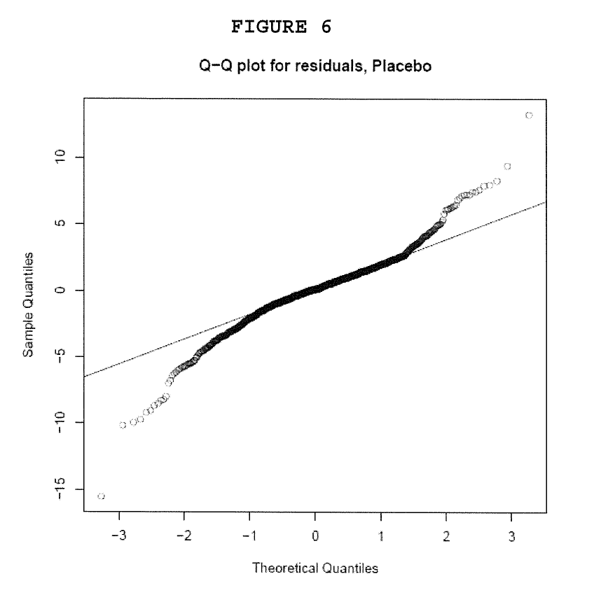

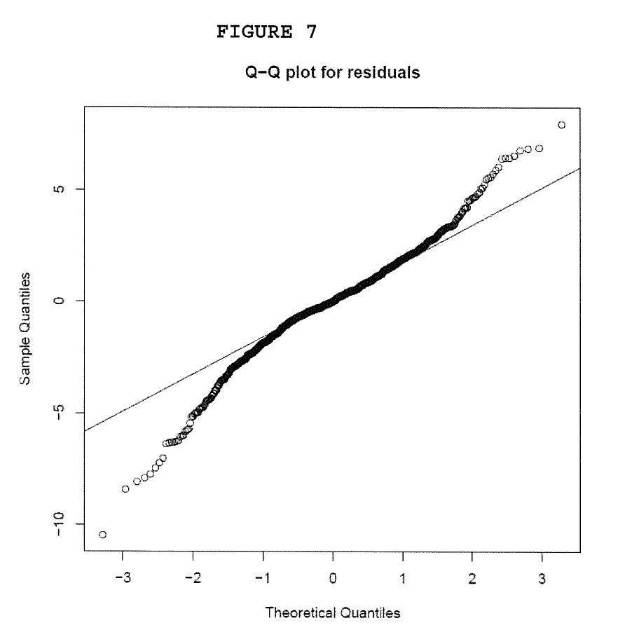

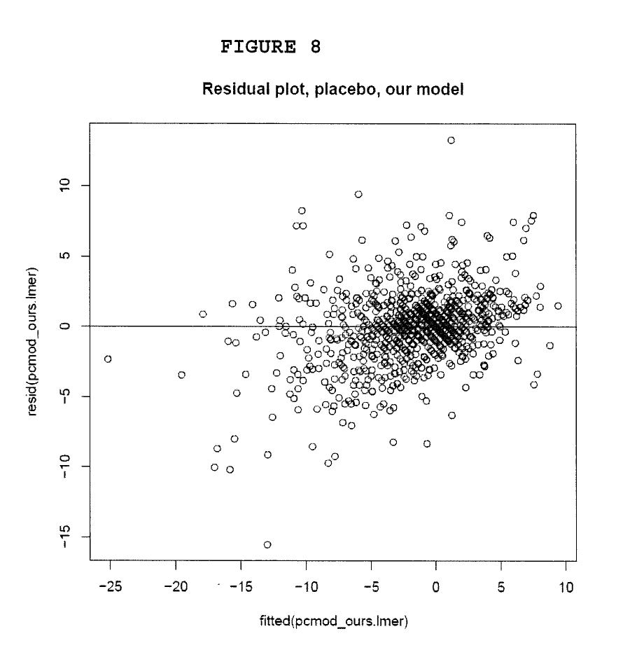

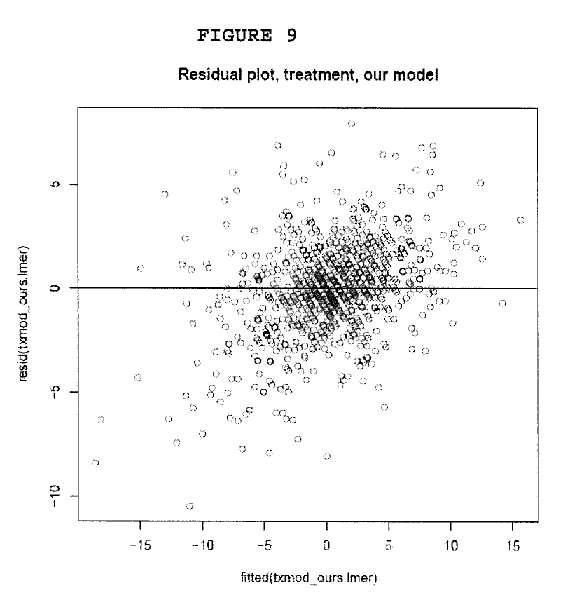

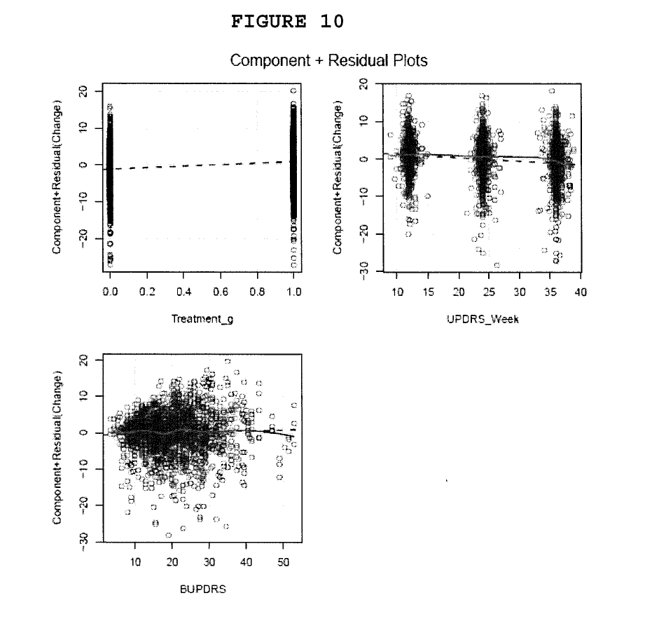

FIGS. 6-10 display the QQ plots and the residuals of the model that are described in the model building section.

### Model Building

If an underlying assumption of a linear trend for the trajectory of the response over time is made, a continuous variable for time is appropriate and the coefficient of the variable is slope; otherwise a categorical time variable can be fit. Linearity can be determined visually. The trajectory was inspected using a model of the fixed effects:

Change=Treatment+UPDRS_Week+Treatment*UPDRS_Week+TimeDiag+BUPDRS+age+Tobacco+Country, Model (1)

where Treatment represented either being on placebo or active treatment, UPDRS_Week represented the time of the study in weeks, Treatment*UPDRS_Week was the interaction of these two effects, TimeDiag was the time since diagnosis of Parkinson's Disease, BUPDRS was the baseline UPDRS, age was the age of the individual when they entered the study, Tobacco represented smoking status and Country indicated residency.

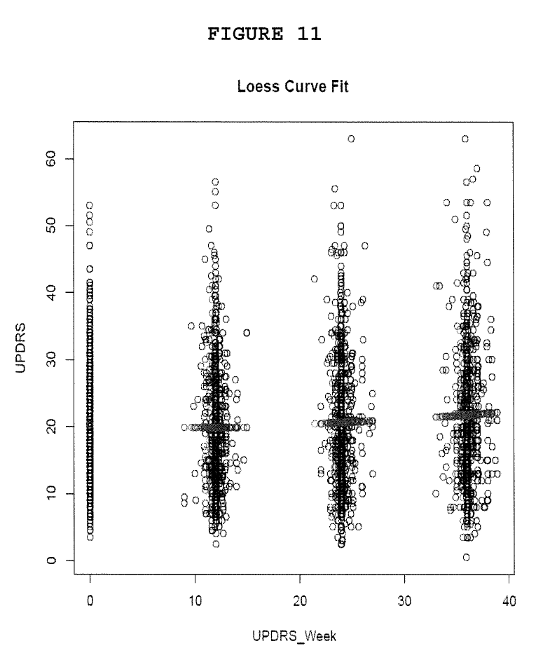

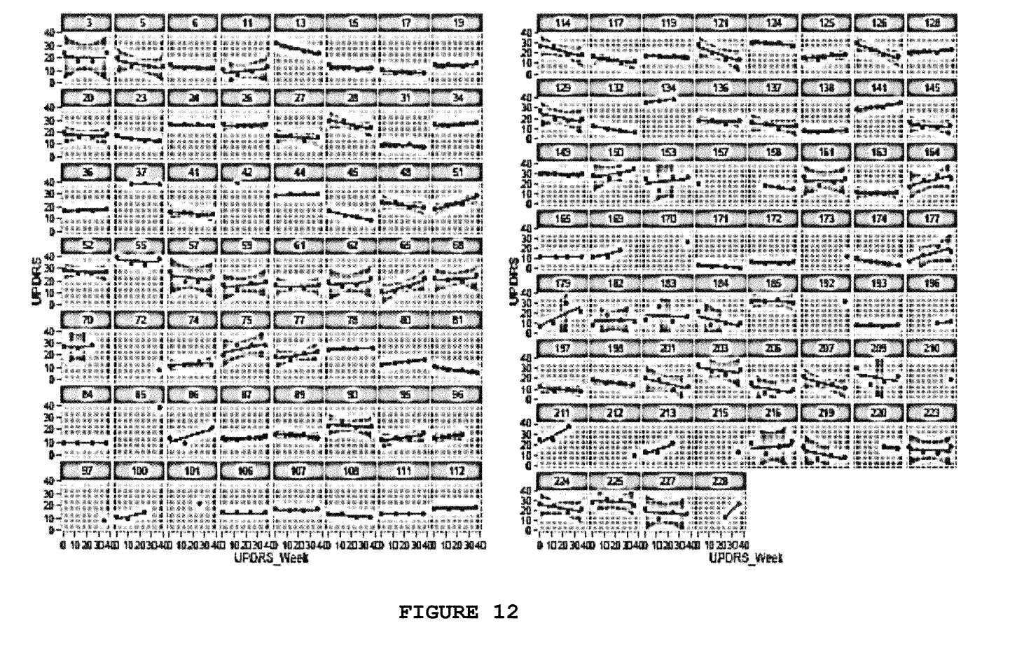

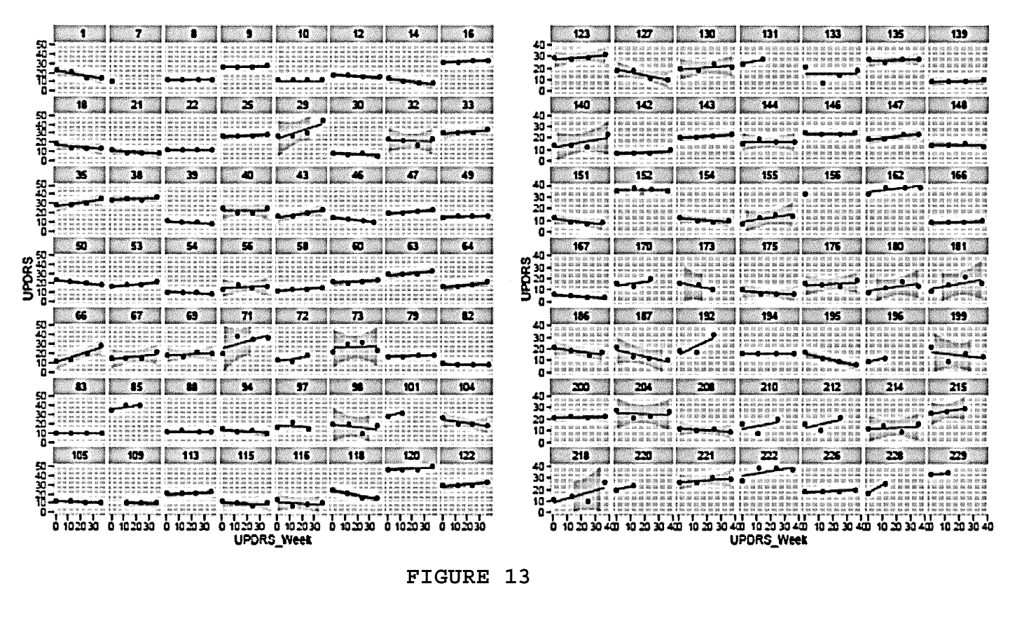

FIGS. 11-13: Model Building helps to investigate the trend of the data over time; UPDRS over time appeared to be linear suggesting that a quantitative time is preferred over a categorical variable.

The Clarke test is a statistical method for testing non-nested models. In this case it was used to test quantitative versus categorical time. The Clarke test was applied to the two models (i.e., quantitative vs. categorical time). A log-likelihood value for each model and a test statistic were derived. The two-sided distribution-free Clarke test looks at the difference in the model medians and derives a p-value from there [Clarke 2007; Vuong 1989]. The Clarke test was completed on linear models of categorical time versus the model with quantitative time. The models used for these tests were the same as in Model (1). However, one test used a fixed effect model with UPDRS_Week as a quantitative input, while the other model used a categorical time value. The categorical time assigned the raw time values into the closest category of 12 weeks, 24 weeks or 36 weeks.

The results in Table 1 indicate that neither model was preferred. Thus, time was modeled linearly as a quantitative variable rather than as a categorical variable. Model building was therefore continued with UPDRS_Week as a quantitative variable.

TABLE 1
Clarke test. Test to see if a model with categorical
time or quantitative time is preferred

| Clarke test for non-nested models | Value |
|---|---|
| Categorical time log-likelihood | −5377 |
| Quantitative time log-likelihood | −5377 |
| Observations | 1785 |
| Test statistic | 912 (51%) |
| Neither model is significantly preferred | (p = 0.37) |

### Covariates

A fit to the maximum model was needed to fit a ‘good enough’ model. In this case, a mixed model that includes all of the variables in Model (1), using UPDRS_Week as a quantitative variable, and including a random intercept for individual, which is standard practice, was looked at and in addition included “week” as a random slope. TevalDn was a variable that holds the subject number identifier to avoid mixing data from different subjects.

Change=Treatment*UPDRS_Week+TimeDiag+BUPDRS+age+Tobacco+Country+(1|TevaIDn)+(1|UPDRS_Week) Model (2)

The model was tested by a type III test with Kenward-Rogers denominator degrees of freedom with results in Table 2.

TABLE 2
Analysis of Variance Table with Kenward-Roger approximation for degrees of freedom
for Model (2) with Treatment considered as either on Placebo or Treatment.

| | | Sum | Mean | F | | Pr | |
|---|---|---|---|---|---|---|---|
| Covariate | Df | Sq | Sq | value | Denom | (>F) | Significance |
| Treatment g | 1 | 177 | 177 | 1 | 1290.91 | 0.84541 | |
| UPDRS_Week | 1 | 663 | 663 | 1 | 41.68 | 3.653e−11 | *** |
| TimeDiag | 1 | 426 | 426 | 1 | 455.55 | 0.74705 | |
| BUPDRS | 1 | 35567 | 35567 | 1 | 474.96 | <2.2e−16 | *** |
| Age | 1 | 1 | 1 | 1 | 459.27 | 0.93633 | |
| Tobacco g | 2 | 16 | 8 | 1 | 458.94 | 0.28357 | |
| Country | 8 | 218 | 27 | 1 | 467.68 | 0.07831 | . |
| Treatment g: | 1 | 497 | 497 | 1 | 1369.01 | 1.688e−08 | *** |
| UPDRS Week | | | | | | | |

The results of this model suggested that not all of the covariates included were necessary. In fact only UPDRS_Week, BUPDRS, and the interaction of Treatment by Week were significant, the main effect of treatment is automatically included.

### Random Effects

In addition to testing the fixed effects, the random effects of this model were also tested with the “rand” function in the lmerTest package (http://cran.r-project.org/web/packages/lmerTest/lmerTest.pdf), Table 3. The significance test of the random effects from the imerTest package performed a log-likelihood ratio test on the random effects and returned a data frame of χ² test statistics with the corresponding p-values. The random effects analysis for Model (2) is presented in the following table. Both random effects were highly significant.

TABLE 3
Analysis of Random effects of the Model

| | | | | Signif- |
|---|---|---|---|---|
| Covariate | Chi.sq | Chi.DF | p.value | icance |
| (1 \| TevaIDn) | 250.4 | 1 | <2e−16 | *** |
| (1 \| UPDRS_Week) | 12.2 | 1 | 5e−04 | *** |

A reduced model including only the significant variables was tested with time as categorical and quantitative using the Clarke test with results in Table 4, show that the Quantitative time model is preferred with a significant p-value, p<0.05. A quantitative time variable for a reduced model was therefore pursued, which is the model that includes only the significant covariates as indicated by the model fitting above.

TABLE 4
Clarke test to verify the time variable as categorical or quantitative.
Quantitative time is preferred with a significant p-value.

| Clarke test for non-nested models | Value | |
|---|---|---|
| Categorical time log-likelihood | −6611 | |
| Quantitative time log-likelihood | −6611 | |
| Observations | 2094 | |
| Test statistic | 963 | (46%) |
| Quantitative time model is preferred | (p = | 0.00026) |

### Random Slope

A random slope model was tested, as a random intercept model is anti-conservative with a higher type I error rate. Generally, individuals can differ in their response to experimental manipulation, therefore random slopes may be more appropriate as it is not expected that the effect of treatment be the same for each individual or item, (UPDRS_Week).

To complete a random slope model, random effects as intercepts, as in Model (2), must not be highly correlated. The correlation of the random effects of Model (2) was tested, Table 5.

TABLE 5
Results of Random Slope model fit for Model (3).

| Groups | Name | Variance | Std Dev | Corr |
|---|---|---|---|---|
| TevaIDn | (Intercept) | 56.740120 | 7.53260 | |
| | UPDRS_Week | 0.016813 | 0.12966 | 0.368 |
| Residual | | 11.612670 | 3.40774 | |

There were 691 individuals. Therefore there was not perfect correlation in this model.

The random slope model tested is:

Change=Treatment*UPDRS_Week+(1+UPDRS_Week|TevaIDn). Model (3)

The significance of the random effects for Model (3) was tested. The test was performed by the rand function of the imerTest package for Model (3), Table 6.

TABLE 6
Significance of random effects for Model (8). The random
slope term was highly significant for this model.

| | | | | Signif- |
|---|---|---|---|---|
| Covariate | Chi.sq | Chi.DF | p.value | icance |
| (1 + UPDRS_Week \| TevaIDn) | 35.8 | 1 | 2e−09 | *** |

After testing the association between the random effects, Table 6, a high correlation was not observed. The random slope is also highly significant, Table 6. The random slope model was preferred.

For this study each of 7 analyses, were completed for both models.

### Analysis 1

The first analysis was the analysis of UPDRS during the placebo control phase, N=692. This analysis included all individuals during the first 36 weeks of the study with approximately half of the individuals being on treatment and half on placebo. The change in UPDRS from baseline at each time point was examined. The null hypothesis was that there will be no interaction effect of treatment by week by genotype. Using a type III test with Kenward-Rogers denominator degrees of freedom, it was not possible to reject the null hypothesis.

### Analysis 2

This analysis was completed with only the delayed start individuals, N=333. The data from each visit was used in the analysis so there were UPDRS recordings for weeks 0 (Baseline), 12, 24, 36, 42, 48, 54, 60, 66, and 72. Again, looking at the change in the UPDRS from baseline for the null hypothesis of no Phase by week by genotype interaction, it was not possible to reject the null hypothesis for either model.

### Analysis 3

For this analysis, all individuals while on treatment only, N=677, that is, combined analysis of the early start group and the delayed start group on active treatment, were used. For the delayed start group only 4 UDPRS readings at equivalent time points to the readings of the early start group were used. 0 (baseline), 12, 24 and 36 for the early start group and 36 (baseline), 48, 60 and 72 for the delayed start group were used. This analysis did not include placebo data. For this analysis, it was not possible to reject the null hypothesis of no week by genotype interaction for either of the models developed above.

This analysis was repeated including dosage as a covariate in the model. With the inclusion of the dose variable, it was not possible to reject the null hypothesis. In summary, there was no genotype by treatment effect.

### Analysis 4

This analysis examined sustained improvement over 36 weeks. It was a Cox proportional hazards model. The data from the first 36 weeks of all individuals who completed 36 weeks of treatment were used, N=634. Sustained improvement was defined as an improvement (i.e., reduction) in UPDRS score of 3.5 or more that is first observed at either 12 or 24 weeks and persisted at 24 or 36 weeks, respectively. For this analysis, using a Type III test, it was not possible to reject the null hypothesis of no Treatment by Genotype interaction.

### Analysis 5

The analysis examined for genetic association with Peak Motor Benefit, which was defined as the greatest improvement at 12, 24, or 36 weeks. Individuals were included in this test if they completed at least 12 weeks of the study, N=682. This analysis was first completed using data from the early start group and from those on placebo, and was replicated in the delayed start group if a positive result was found. For this analysis, individuals were separated into groups of non-responders, intermediate responders and super-responders based on the greatest improvement displayed at any time point during treatment. This was completed by dividing the population into tertiles. The non-responders have a peak motor benefit of less than 4 point improvement, the intermediate responders have a peak motor benefit of between 4 and 12 points and the super responders have a peak motor benefit improvement of more than 12 points. The comparisons were then completed as non-responders versus the rest and super-responders versus the rest. It was found that for both tests, it was not possible to reject the null hypothesis of no treatment by genotype effect for either model.

A similar analysis was repeated by separating the population into two groups based on the criteria of minimally significant clinical change. The groups were those with a peak motor benefit of less than 3.5 reduction in UPDRS scores and those with a peak motor benefit of 3.5 or more. Again, it was not possible to reject the null hypothesis for either model.

### Analysis 5A

The analysis examined for genetic association with Peak Motor Benefit, which was defined as the greatest improvement at 12, 24, or 36 weeks. Individuals were included in this test if they completed at least 12 weeks of the study, N=682. This analysis was first completed using data from the early start group and from those on placebo, and was replicated in the delayed start group if a positive result was found. For this analysis, individuals were separated into groups of non-responders, intermediate responders and super-responders based on the greatest improvement displayed at any time point during treatment. This was completed by dividing the population into tertiles. The non-responders have a peak motor benefit of less than 4 point improvement, the intermediate responders have a peak motor benefit of between 4 and 12 points and the super responders have a peak motor benefit improvement of more than 12 points. The comparisons were then completed as non-responders versus the rest and super-responders versus the rest. It was found that for both tests, it was not possible to reject the null hypothesis of no treatment by genotype effect for either model.

A similar analysis was repeated by separating the population into two groups based on the criteria of minimally significant clinical change. The groups were those with a peak motor benefit of less than 3.5 reduction in UPDRS scores and those with a peak motor benefit of 3.5 or more. Again, it was not possible to reject the null hypothesis for either model.

### Analysis 6

### Change in UPDRS Scores at 12, 24, 36 Weeks

This model measured the change in UPDRS from baseline to 12 weeks in both the early and delayed start group, N=679. The model used for this analysis was

ΔUPDRS=Treatment_g*snps+TimeDiag+BUPDRS+agepc+Country,

where Treatment_g represented either early (active treatment) or delayed start (placebo), SNPs represented the markers, TimeDiag represented the time since diagnosis, BUPDRS was the baseline score, agepc was the age when entering the trial and Country was the location the patients resided during the trial. There were 4 significant results identified that survived correction for multiple testing using the False Discovery Rate (FDR). The SNPs are listed in Table 7, with the p-values found in this analysis. In summary, there was a significant finding for these 4 SNPs using the 12 week data which controls for the placebo effect.

TABLE 7
12 week SNPs. SNPs that were found to have a significant FDR p-value.

| SNP_allele | Unadjusted | Bonferroni | Nyholt | FDR |
|---|---|---|---|---|
| rs1076560_A | 0.0007057301 | 0.1347944548 | 0.07614 | 0.0449314849 |
| rs2283265_A | 0.0006030656 | 0.1151855334 | 0.06512 | 0.0449314849 |
| rs1079597_T | 0.002181838 | 0.4167310612 | 0.216 | 0.0868429524 |
| rs36023_A | 0.000448514 | 0.0856661814 | 0.04844 | 0.0449314849 |

The analysis of the 24 and 36 week data in a similar fashion represented longer term or more persistent effects of rasagiline compared to placebo. This analysis included N=679 datapoints and was completed using the same model above, except with these time points. The null hypothesis was not rejected, that is, no effect of treatment by genotype was found.

Dose was added as a covariate to the 12 week model and after this addition, the null hypothesis could not be rejected. There was no treatment by genotype effect.

### Linkage Disequilibrium (LD) Analysis

Three of the four positive results found in the 12 week change in UPDRS test were from SNPs within the dopamine D2 receptor gene (DRD2). The LD structure was checked in PLINK for these SNPs. As the pairwise r² values were all >0.95 conditional analysis was not undertaken. The correlation matrix for r² for the SNPs in DRD2 is in Table 8.

TABLE 8
Correlation matrix for DRD2 LD.

| | rs1076560 | rs2283265 | rs1079597 |
|---|---|---|---|
| rs1076560 | 1 | 0.956 | 0.953 |
| rs2283265 | 0.956 | 1 | 1 |
| rs1079597 | 0.953 | 1 | 1 |

As shown from the above LD results, 3 of the SNPs that were found to be significant in the 12 week analysis were in high LD with each other and were localized within DRD2. It was concluded that this was a single signal identified by many SNPs. The frequencies of the SNPs in the above correlation matrix are in Table 9.

The fourth SNP rs36023 that was identified to be associated with 12 week change in UPDRS scores was located within the gene encoding the Norepinephrine Transporter (SLC6A2).

TABLE 9
SNP information. Information about the SNPs with
the positive results in the 12 week analysis.

| | Major | Minor | Hom. | | Hom | |
|---|---|---|---|---|---|---|
| SNP | Allele | Allele | Major | Heter | Minor | MAF |
| rs1076560_A | C | A | 502 | 200 | 26 | 0.1731 |
| rs2283265_A | C | A | 486 | 177 | 24 | 0.1638 |
| rs1079597_T | C | T | 503 | 194 | 24 | 0.1678 |
| rs36023_A | G | A | 284 | 356 | 91 | 0.368 |

### Analysis 7

### Change in UPDRS Scores at 12, 24, 36 Weeks

This model measured the change in UPDRS from baseline to 12 weeks in both the early and delayed start group, N=679. The model used for this analysis was

ΔUPDRS=Treatment_g*snps+BUPDRS,

where Treatment_g represented either early (active treatment) or delayed start (placebo, SNPs represented the markers, BUPDRS was the baseline score. There were 2 significant results identified that survived correction for multiple testing using the False Discovery Rate (FDR). The SNPs are listed in Table 10, with the p-values found in this analysis. In summary, there was a significant finding for these 2 SNPs using the 12 week data which controls for the placebo effect.

TABLE 10
12 week SNPs. SNPs that were found
to have a significant FDR p-value.

| SNP_allele | Unadjusted | FDR |
|---|---|---|
| rs1076560_A | 0.00030993 | 0.03006345 |
| rs2283265_A | 0.00023084 | 0.03006345 |

The analysis of the 24 and 36 week data in a similar fashion represented longer term or more persistent effects of rasagiline compared to placebo. This analysis included N=684 datapoints and was completed using the same model above, except with these time points. The null hypothesis was not rejected, that is, no effect of treatment by genotype was found.

Dose was added as a covariate to the 12 week model and after this addition, the null hypothesis could not be rejected. There was no treatment by genotype effect.

### Linkage Disequilibrium (LD) Analysis

The two positive results found in the 12 week change in UPDRS test were from SNPs within the dopamine D2 receptor gene (DRD2). The LD structure was checked in PLINK for these SNPs. As the pairwise r² values were all >0.95 conditional analysis was not undertaken. The correlation matrix for r² for the SNPs in DRD2 is in Table 11.

TABLE 11
Correlation matrix for DRD2 LD.

| | rs1076560 | rs2283265 |
|---|---|---|
| rs1076560 | 1 | 0.956 |
| rs2283265 | 0.956 | 1 |

As shown from the above LD results, the SNPs that were found to be significant in the 12 week analysis were in high LD with each other and were localized within DRD2. It was concluded that this was a single signal identified by many SNPs. The frequencies of the SNPs in the above correlation matrix are in Table 12.

TABLE 12
SNP information. Information about the SNPs with
the positive results in the 12 week analysis.

| | Major | Minor | Hom. | | Hom | |
|---|---|---|---|---|---|---|
| SNP | Allele | Allele | Major | Heter | Minor | MAF |
| rs1076560_A | C | A | 502 | 200 | 26 | 0.1731 |
| rs2283265_A | C | A | 486 | 177 | 24 | 0.1638 |

Summary of the Effect Findings in the Adagio PGX Study According to analysis 6

Two SNPs in tight linkage disequilibrium within the dopamine D2 receptor gene (DRD2) were found to be significantly associated with peak change in UPDRS scores at 12 weeks (rs1076560 and rs2283265, False Discovery Rate [FDR]-corrected p=0.045 for each).

Change in UPDRS from Baseline to Week 12 was −2.19±0.56 for Azilect® treated subjects with rs1076560 CC genotype (N=228, 32.8%), 0.15±0.65 for other Azilect® subjects (N=114, 16.4%), and −0.31±0.53 for Placebo subjects (N=353, 50.8%). See Table 13.

TABLE 13
Least Squares Means of UPDRS change from
Baseline to Week 12 for rs1076560 variants

| Group | LS Mean | Standard Error | Pr > \|t\| |
|---|---|---|---|
| Azilect ® CC | −2.18986600 | 0.56487917 | 0.0001 |
| Azilect ® other | 0.15063502 | 0.64970361 | 0.8167 |
| Placebo | −0.30783632 | 0.52926725 | 0.5610 |

These results reflected a −2.34 UPDRS advantage for Azilect® treated subjects with CC genotype over the other Azilect® subjects (95% CI: −3.40-−1.28), and −1.88 UPDRS advantage for Azilect® treated subjects with CC genotype over Placebo subjects (95% CI: −2.68-−1.08). The difference between Azilect® subjects with AA or AC genotype and Placebo subjects is 0.46 and is not statistically significant (95% CI: −0.54-1.46). See Table 14.

TABLE 14
Least Squares Means of UPDRS change from
Baseline to Week 12 for rs1076560 variants

| | | Difference | 95% Confidence | |
|---|---|---|---|---|
| | | Between | Limits for | |
| Group 1 | Group 2 | LS Means | Difference | |
| Azilect ® CC | Azilect ® other | −2.340501 | −3.402699 | −1.278303 |
| Azilect ® CC | Placebo | −1.882030 | −2.679296 | −1.084764 |
| Azilect ® other | Placebo | 0.458471 | −0.544543 | 1.461486 |

Results for rs2283265 were almost identical, as the two SNPs are in Linkage Disequilibrium. See tables 15-16.

TABLE 15
Least Squares Means of UPDRS change from
Baseline to Week 12 for rs2283265 variants

| | | Standard | |
|---|---|---|---|
| Group | LS Mean | Error | Pr > \|t\| |
| Azilect ® CC | −2.25210668 | 0.56871205 | <.0001 |
| Azilect ® other | 0.06118556 | 0.63624926 | 0.9234 |
| Placebo | −0.31285867 | 0.52910093 | 0.5545 |

TABLE 16
Least Squares Means of UPDRS change from
Baseline to Week 12 for rs2283265 variants

| | | Difference | 95% Confidence | |
|---|---|---|---|---|
| | | Between | Limits for | |
| Group 1 | Group 2 | LS Means | Difference | |
| Azilect ® CC | Azilect ® other | −2.313292 | −3.352322 | −1.274263 |
| Azilect ® CC | Placebo | −1.939248 | −2.746427 | −1.132069 |
| Azilect ® other | Placebo | 0.374044 | −0.597565 | 1.345654 |

A third SNP within the gene for the Norepinephrine Transporter (SLC6A2) was also found to be associated with this endpoint (rs36023, FDR-corrected p=0.045).

Change in UPDRS from Baseline to Week 12 was −1.37±0.54 for Azilect® treated subjects (N=342, 49.2%), 1.70±0.0.94 for Placebo subjects with AA genotype (N=38, 5.5%), and −0.31±0.53 for the other Placebo subjects (N=315, 45.3%). See Table 17.

TABLE 17
Least Squares Means of UPDRS change from
Baseline to Week 12 for rs36023 variants

| | | Standard | |
|---|---|---|---|
| Group | LS Mean | Error | Pr > \|t\| |
| Azilect ® | −1.37423962 | 0.54214111 | 0.0115 |
| Placebo AA | 1.70266991 | 0.93693222 | 0.0696 |
| Placebo other | −0.52242319 | 0.53850225 | 0.3323 |

These results reflected a 2.23 UPDRS disadvantage for Placebo treated subjects with AA genotype compared to the other Placebo subjects (95% CI: 0.57-3.88), and 3.08 UPDRS disadvantage for Placebo treated subjects with AA genotype when compared to Azilect®subjects (95% CI 1.44-4.71). Azilect® subjects also had 0.85 UPDRS advantage over Placebo subjects with AG or GG genotype (95% CI: 0.11-1.59). See Table 18.

TABLE 18
Least Squares Means of UPDRS change from
Baseline to Week 12 for rs36023 variants

| | | Difference | 95% Confidence | |
|---|---|---|---|---|
| | | Between | Limits for | |
| Group 1 | Group 2 | LS Means | Difference | |
| Azilect ® | Placebo AA | −3.076910 | −4.712804 | −1.441015 |
| Azilect ® | Placebo other | −0.851816 | −1.593505 | −0.110127 |
| Placebo AA | Placebo other | 2.225093 | 0.574701 | 3.875486 |

### Summary of the Effect Findings in the Adagio PGX Study According to Analysis 7

Two SNPs in tight linkage disequilibrium within the dopamine D2 receptor gene (DRD2) were found to be significantly associated with peak change in UPDRS scores at 12 weeks (rs1076560 and rs2283265, False Discovery Rate [FDR]-corrected p=0.030 for each).

Change in UPDRS from Baseline to Week 12 was −1.68±0.31 for Azilect® treated subjects with rs1076560 CC genotype (N=231, 33.4%), 0.64±0.44 for other Azilect® subjects (N=114, 16.5%), and −0.24±0.26 for Placebo subjects (N=347, 50.1%). See Table 19.

TABLE 19
Least Squares Means of UPDRS change from
Baseline to Week 12 for rs1076560 variants

| | | Standard | |
|---|---|---|---|
| Group | LS Mean | Error | Pr > \|t\| |
| Azilect ® CC | −1.67885538 | 0.30870113 | <.0001 |
| Azilect ® other | 0.64318840 | 0.43957948 | 0.1439 |
| Placebo | 0.23807177 | 0.25629332 | 0.3533 |

These results reflected a −2.32 UPDRS advantage for Azilect® treated subjects with CC genotype over the other Azilect®subjects (95% CI: −3.38-−1.27), and −1.92 UPDRS advantage for Azilect® treated subjects with CC genotype over Placebo subjects (95% CI: −2.71-−1.28). The difference between Azilect=subjects with AA or AC genotype and Placebo subjects is 0.41 and is not statistically significant (95% CI: −0.60-1.41). See Table 20.

TABLE 20
Least Squares Means of UPDRS change from
Baseline to Week 12 for rs1076560 variants

| | | Difference | 95% Confidence | |
|---|---|---|---|---|
| | | Between | Limits for | |
| Group 1 | Group 2 | LS Means | Difference | |
| Azilect ® CC | Azilect ® other | −2.322044 | −3.375530 | −1.268557 |
| Azilect ® CC | Placebo | −1.916927 | −2.705930 | −1.127924 |
| Azilect ® other | Placebo | 0.405117 | −0.595530 | 1.405763 |

Results for rs2283265 were almost identical, as the two SNPs are in Linkage Disequilibrium. See tables 21-22.

TABLE 21
Least Squares Means of UPDRS change from
Baseline to Week 12 for rs2283265 variants

| | | Standard | |
|---|---|---|---|
| Group | LS Mean | Error | Pr > \|t\| |
| Azilect ® CC | −1.72895922 | 0.31636586 | <.0001 |
| Azilect ® other | 0.52691779 | 0.41990164 | 0.2100 |
| Placebo | 0.23811400 | 0.25634426 | 0.3533 |

TABLE 22
Least Squares Means of UPDRS change from
Baseline to Week 12 for rs2283265 variants

| | | Difference | 95% Confidence | |
|---|---|---|---|---|
| | | Between | Limits for | |
| Group 1 | Group 2 | LS Means | Difference | |
| Azilect ® CC | Azilect ® other | −2.255877 | −3.286985 | −1.224769 |
| Azilect ® CC | Placebo | −1.967073 | −2.767748 | −1.166399 |
| Azilect ® other | Placebo | 0.288804 | −0.678728 | 1.256336 |

### X Chromosome Analyses

The X chromosome was analyzed a second time for the above tests. The second analysis was completed by counting the number of alleles each individual has; males have either 0 or 1 allele and females have 0, 1, or 2 copies. In the above analysis, the number of alleles for males were doubled, they had either 0 or 2 copies, to account for x inactivation as in [4].

After recoding the SNPs on the X chromosome, the null hypothesis was not rejected for any of the tests.

### Adagio PGx Post Hoc Analysis Description and Results

### Description of Analyses

Following the analyses described in the Statistical Analysis of the ADAGIO PGx study, 3 additional analyses were performed in a post hoc manner, to explore the association between short term change in UPDRS, treatment by Azilect and genotype.

The 3 analyses were performed on 3 response variables:
Change in UPDRS from baseline to week 12
Change in UPDRS from baseline to Week 24
Change in UPDRS from baseline to week 36

The analysis utilized an Analysis of Covariance (ANCOVA) model (SAS PROC GLM).

The models included the following effects:
Treatment group—Placebo (1 mg delayed start and 2 mg delayed start groups combined) or Azilect® (1 mg early start and 2 mg early start groups combined).
Genotype
Treatment by Genotype interaction
Time from PD diagnosis
Baseline Total UPDRS score
Age at baseline
Smoking status—Current smoker: Yes/No. This covariate will be included in the model only for CYP1A2 markers.
Country

Multiplicity of tested hypotheses was controlled using FDR.

### Results

4 statistically significant treatment by genotype interactions were detected, all with regard to change in UPDRS from baseline to week 12. A summary of the results are presented in Table 23 below:

TABLE 23
Analysis of Change in UPDRS Score from Baseline to Weeks 12, 24 and 36; Test of Treatment by Genotype Effect;

| | Change from Baseline to Week 12 | | | Change from Baseline to Week 12 | | | Change from Baseline to Week 36 | | |
|---|---|---|---|---|---|---|---|---|---|
| | | FDR | Bonferroni | | FDR | Bonferroni | | FDR | Bonferroni |
| | Unadjusted | Adjusted | Adjusted | Unadjusted | Adjusted | Adjusted | Unadjusted | Adjusted | Adjusted |
| Marker | P-value | P-value | P-value | P-value | P-value | P-value | P-value | P-value | P-value |
| DRD2_rs1076560 | 0.0003 | 0.0207 | 0.0503 | 0.2688 | 0.9796 | 1.0000 | 0.8130 | 0.9844 | 1.0000 |
| DRD2_rs1079597 | 0.0009 | 0.0422 | 0.1687 | 0.3140 | 0.9796 | 1.0000 | 0.9134 | 0.9892 | 1.0000 |
| DRD2_rs2283265 | 0.0002 | 0.0207 | 0.0366 | 0.2386 | 0.9796 | 1.0000 | 0.6923 | 0.9754 | 1.0000 |
| SLC6A2_{——}rs36023 | 0.0003 | 0.0207 | 0.0620 | 0.0713 | 0.9796 | 1.0000 | 0.0326 | 0.4939 | 1.0000 |

The 3 SNPs detected on the DRD2 gene appear to be in LD.

### Results for Marker DRD2_rs1079597_n

TABLE 24
Adjusted means and Standard Deviations for marker
DRD2_rs1079597_n by treatment group

| | | Adjusted | Standard |
|---|---|---|---|
| Treatment | DRD2_rs1079597_n | Mean | Error |
| Azilect | 0 (TT) | 0.41064072 | 1.65925735 |
| Azilect | 1 (CT) | 0.17026989 | 0.67358646 |
| Azilect | 2 (CC) | −2.13953356 | 0.56977996 |
| Placebo | 0 (TT) | −1.39531300 | 1.40019443 |
| Placebo | 1 (CT) | −0.36643801 | 0.71279800 |
| Placebo | 2 (CC) | −0.25876812 | 0.55839195 |

TABLE 25
Treatment Effect within genotype level -
DRD2_rs1079597_n (Azilect - Placebo)

| | | Standard | t | Pr > |
|---|---|---|---|---|
| Genotype | Estimate | Error | Value | \|t\| |
| DRD2_rs1079597_n = | 1.80595373 | 2.06238172 | 0.88 | 0.3815 |
| 0 (TT) | | | | |
| DRD2_rs1079597_n = | 0.53670790 | 0.70983388 | 0.76 | 0.4499 |
| 1 (CT) | | | | |
| DRD2_rs1079597_n = | −1.88076543 | 0.44584725 | −4.22 | <.0001 |
| 2 (CC) | | | | |

The results indicate that patients with CC genotype have 1.88 UPDRS advantage when treated by Azilect, when compared to untreated patients with CC genotype.

### Results for Marker DRD2_rs2283265_n

TABLE 26
Adjusted means and Standard Deviations for marker
DRD2_rs2283265_n by treatment group

| | | Adjusted | Standard |
|---|---|---|---|
| Treatment | DRD2_rs2283265_n | Mean | Error |
| Azilect | 0 (AA) | 2.30669115 | 1.71407493 |
| Azilect | 1 (AC) | 0.03216449 | 0.68812319 |
| Azilect | 2 (CC) | −2.16978016 | 0.56980452 |
| Placebo | 0 (AA) | −1.55247320 | 1.32745569 |
| Placebo | 1 (AC) | −0.39283168 | 0.71152677 |
| Placebo | 2 (CC) | −0.26950565 | 0.56444646 |

TABLE 27
Treatment Effect within genotype level -
DRD2_rs1079597_n (Azilect - Placebo)

| | | Standard | t | Pr > |
|---|---|---|---|---|
| Genotype | Estimate | Error | Value | \|t\| |
| DRD2_rc2283265_n = | 3.85916434 | 2.05563151 | 1.88 | 0.0609 |
| 0 (AA) | | | | |
| DRD2_rs2283265_n = | 0.42499617 | 0.72240394 | 0.59 | 0.5565 |
| 1 (AC) | | | | |
| DRD2_rs2283265_n = | −1.90027452 | 0.44322584 | −4.29 | <.0001 |
| 2 (CC) | | | | |

The results indicate that patients with CC genotype have 1.90 UPDRS advantage when treated by Azilect, when compared to untreated patients with CC genotype.

### Results for Marker SLC6A2_rs36023_n

TABLE 28
Adjusted means and Standard Deviations for marker
SLC6A2_rs36023_n by treatment group

| | | Adjusted | Standard |
|---|---|---|---|
| Treatment | SLC6A2_rs36023_n | Mean | Error |
| Azilect | 0 (AA) | −1.92777157 | 0.84559648 |
| Azilect | 1 (AG) | −1.83583526 | 0.60740426 |
| Azilect | 2 (GG) | −0.68391490 | 0.63950442 |
| Placebo | 0 (AA) | 1.68800284 | 0.93760416 |
| Placebo | 1 (AG) | −0.16926435 | 0.58479103 |
| Placebo | 2 (GG) | −1.10270130 | 0.64071906 |

TABLE 29
Treatment Effect within genotype level -
SLC6A2_rs36023_n (Azilect - Placebo)

| | | Standard | t | Pr > |
|---|---|---|---|---|
| Genotype | Estimate | Error | Value | \|t\| |
| SLC6A2_rs36023_n = | −3.61577442 | 1.04144019 | −3.47 | 0.0006 |
| 0 (AA) | | | | |
| SLC6A2_rs36023_n = | −1.66657090 | 0.52666194 | −3.16 | 0.0016 |
| 1 (AG) | | | | |
| SLC6A2_rs36023_n = | 0.41878640 | 0.59057395 | 0.71 | 0.4785 |
| 2 (GG) | | | | |

The results indicate that patients with AA genotype have 3.62 UPDRS advantage when treated by Azilect, when compared to untreated patients with AA genotype. In addition, patients with AG genotype have 1.67 UPDRS advantage when treated by Azilect, when compared to untreated patients with AG genotype

### Statistical Analysis of Genomic Effects of Azilect Treatment in the Adagio Trial

The analysis of the ADAGIO PGX data revealed 4 SNPs on two genes that are associated with short term effect of UPDRS change from baseline to week 12.

For the SNPs rs1076560, rs1079597 and rs2283265 within the dopamine D2 receptor gene (DRD2), the analysis reflected an advantage for Azilect treated subjects with CC genotype in those SNPs over the other Azilect subjects and over Placebo treated subjects, while there was not a statistically significant difference between placebo treated subjects and Azilect treated subjects with genotypes AA or AC.

For SNP rs36023 within the gene for the Norepinephrine Transporter (SLC6A2), the analysis reflected a disadvantage for Placebo treated subjects with AA genotype compared to the other Placebo subjects and to the Azilect treated subjects. Azilect treated subjects also had advantage over Placebo treated subjects with AG or GG genotype.

### Example 1

A patient diagnosed with Parkinson's disease provides a DNA sample which is genotyped at SNPs rs1076560 and rs2283265. The subject's genotype is found to be CC at rs1076560 and is identified as a predicted responder to Azilect®. The patient is administered a 1.0 mg dosage of Azilect® and is successfully treated.

### Example 2

A human subject afflicted with Parkinson's disease is administered 2.0 mg of Azilect® daily for 12 weeks and provides a DNA sample for genotyping. The subject's genotype is found to be CC at rs1076560 and rs2283265 and is identified as a predicted responder to Azilect®. Daily administration of Azilect® is continued and the subject is successfully treated.

### Example 3

Three patients diagnosed with Parkinson's disease are genotyped at SNPs rs1076560, rs2283265 and rs36023.

Subject A's genotype is found to be CC at rs1076560 and rs2283265. Subject B's genotype is found to be CC at rs1076560. Subject C's genotype is found to be AA at rs36023 and CC at rs1076560. All three subjects are identified as predicted responders to Azilect® and administered Azilect®. All three subjects are successfully treated.

### Example 4

A male patient with Parkinson's disease is administered Azilect®. The patient provides a DNA sample for genotyping. The patient's genotype is not found to be any of CC at rs1076560 or CC at rs2283265.

The patient is not identified as a predicted responder to Azilect® and Azilect® administration is modified.

### Example 5

A human subject afflicted with Parkinson's disease is administered 1.0 mg of Azilect®. The subject provides a DNA sample for genotyping. The subject's genotype is not found to be CC at rs1076560 or CC at rs2283265.

The subject is administered bromocriptine, benztropine, levodopa, ropinirole, pramipexole, rotigotine, cabergoline, entacapone, tolcapone, amantadine or selegiline.

### Example 6

A sample is collected from a person diagnosed with Parkinson's disease. DNA is extracted from the sample, amplified and applied to a LifeTechnologies OpenArray NT genotyping platform array containing probes for SNPs rs1076560, rs2283265 and rs36023.

The person's genotype is found to be CC at rs1076560 and AA at rs36023. The person is identified as a predicted responder to Azilect® and administered Azilect®. The subject is successfully treated.

### Example 7

A patient diagnosed with Parkinson's disease provides a DNA sample which is genotyped at SNPs rs1079597, rs1076560, and rs2283265. The subject's genotype is found to be CC at rs1079597, and is identified as a predicted responder to Azilect®. The patient is administered a 1.0 mg dosage of Azilect® and is successfully treated.

### Example 8

Three patients diagnosed with Parkinson's disease are genotyped at SNPs rs1076560, rs2283265, rs1079597 and rs36023.

Subject A's genotype is found to be CC at rs1079597, rs1076560 and rs2283265. Subject B's genotype is found to be CC at rs1079597. Subject C's genotype is found to be AA at rs36023 and CC at rs1079597. All three subjects are identified as predicted responders to Azilect® and administered Azilect®. All three subjects are successfully treated.

### Example 9

A male patient with Parkinson's disease is administered Azilect®. The patient provides a DNA sample for genotyping. The patient's genotype is not found to be any of CC at rs1076560 or CC at rs2283265 or CC at rs1079597.

The patient is not identified as a predicted responder to Azilect® and Azilect® administration is modified.

### Example 10

A human subject afflicted with Parkinson's disease is administered 1.0 mg of Azilect®. The subject provides a DNA sample for genotyping. The subject's genotype is not found to be CC at rs1076560 or CC at rs2283265 or CC at rs1079597.

The subject is administered bromocriptine, benztropine, levodopa, ropinirole, pramipexole, rotigotine, cabergoline, entacapone, tolcapone, amantadine or selegiline.

### Example 11

A sample is collected from a person diagnosed with Parkinson's disease. DNA is extracted from the sample, amplified and applied to a LifeTechnologies OpenArray NT genotyping platform array containing probes for SNPs rs1076560, rs2283265, rs1079597 and rs36023.

The person's genotype is found to be CC at rs1079597 and AA at rs36023. The person is identified as a predicted responder to Azilect® and administered Azilect®. The subject is successfully treated.

### REFERENCES

1. A two-stage meta-analysis identifies several new Loci for Parkinson's disease. PLoS Genet 2011; 7.
2. Bar-Am O, Weinreb O, Amit T, Youdim M B. The neuroprotective mechanism of 1-(R)-aminoindan, the major metabolite of the anti-parkinsonian drug rasagiline. J Neurochem 2010; 112: 1131-1137.
3. Chen J J, Ly A V. Rasagiline: A second-generation monoamine oxidase type-B inhibitor for the treatment of Parkinson's disease. Am J Health Syst Pharm 2006; 63: 915-928.
4. Chen J J, Swope D M. Clinical pharmacology of rasagiline: a novel, second-generation propargylamine for the treatment of Parkinson disease. J Clin Pharmacol 2005; 45: 878-894.
5. Clarke K. A simple distribution-free test for nonnested model selection. Political Analysis 2007; 15:3.
6. Do C B, Tung J Y, Dorfman E et al. Web-based genome-wide association study identifies two novel loci and a substantial genetic component for Parkinson's disease. PLoS Genet 2011; 7: e1002141.
7. Nalls M A, Plagnol V, Hernandez D G et al. Imputation of sequence variants for identification of genetic risks for Parkinson's disease: a meta-analysis of genome-wide association studies. Lancet 2011; 377: 641-649.
8. Olanow C W, Rascol O, Hauser R, Feigin P D, Jankovic J, Lang A, Langston W, Melamed E, Poewe W, Stocchi F, Tolosa E; ADAGIO Study Investigators. N Engl J Med. 2009 Sep. 24; 361(13):1268-78. doi: 10.1056/NEJMoa0809335.
9. Purcell S, Neale B, Todd-Brown K et al. PLINK: a tool set for whole-genome association and population-based linkage analyses. Am J Hum Genet 2007; 81:559-575.
10. Vuong Q H. Likelihood ratio tests for model selection and non-nested hypotheses. Econometrica 1989; 57: 333.

1. A method for treating a human subject afflicted with Parkinson's disease (PD) with a pharmaceutical composition comprising rasagiline or a pharmaceutically acceptable salt of rasagiline, and a pharmaceutically acceptable carrier, comprising the steps of:
(i) obtaining a biological sample comprising a genome from the human subject afflicted with Parkinson's disease;
(ii) assaying the DNA or RNA of the biological sample from the human subject using a probe or a primer, to determine the diploid genotype of the human subject at single nucleotide polymorphism (SNP) rs1076560 or rs2283265;
(iii) identifying the human subject as a predicted responder to rasagiline if the diploid genotype is CC at rs1076560, CC at rs2283265, or CC at both rs1076560 and rs2283265; and
(iv) administering the pharmaceutical composition comprising rasagiline and a pharmaceutically acceptable carrier to the human subject if the human subject is identified as a predicted responder to rasagiline.

2-5. (canceled)

6. The method of claim 1, wherein the pharmaceutical composition comprising rasagiline and a pharmaceutically acceptable carrier is administered as monotherapy.

7. The method of claim 1, wherein the pharmaceutical composition comprising rasagiline and a pharmaceutically acceptable carrier is administered in combination at least one other Parkinson's disease drug.

8. The method of claim 1, wherein step iv) further comprises administering a pharmaceutical composition which does not comprise rasagiline to the subject if the subject is not a predicted responder.

9. The method of claim 8, wherein in the human subject is administered a pharmaceutical composition comprising bromocriptine, benztropine, levodopa, ropinirole, pramipexole, rotigotine, cabergoline, entacapone, tolcapone, amantadine or selegiline and a pharmaceutically acceptable carrier if the subject is not identified as a responder.

10. A method for treating a human subject afflicted with Parkinson's disease comprising the steps of:
(i) administering to the human subject a therapeutic amount of a pharmaceutical composition comprising rasagiline, or a pharmaceutically acceptable salt of rasagiline, and a pharmaceutically acceptable carrier;
(ii) obtaining a biological sample comprising a genome from the human subject afflicted with Parkinson's disease;
(iii) assaying the DNA or RNA of the biological sample from the human subject using a probe or a primer, to determine the diploid genotype of the human subject at single nucleotide polymorphism (SNP) rs1076560 or rs2283265;
(iv) identifying the human subject as a predicted responder to rasagiline if the diploid genotype is CC at rs1076560, CC at rs2283265, or CC at both rs1076560 and rs2283265; and
(v) continuing administration of the pharmaceutical composition if the human subject is identified as a predicted responder to rasagiline, or modifying the administration of the pharmaceutical composition to the human subject if the human subject is not identified as a predicted responder to rasagiline.

11. The method of claim 10, wherein step ii) is conducted 12, 24, or 36 weeks after initiation of administration of rasagiline or a pharmaceutically acceptable salt of rasagiline.

12. The method of claim 11, wherein step ii) is conducted 12 weeks after initiation of administration of rasagiline or a pharmaceutically acceptable salt of rasagiline.

13. The method of claim 10, wherein a predicted responder's rate of improvement of Parkinson's disease is quantified by the Total UPDRS score, wherein a sustained improvement is a reduction in UPDRS score of 3.5 or more than is first observed at either 12 or 24 weeks and persisted at 24 or 36 weeks, respectively.

14. The method of claim 1, comprising identifying the human subject as a predicted responder to rasagiline for a period of more than 12 weeks, more than 24 weeks, or more than 36 weeks.

15. The method of claim 1, wherein the pharmaceutically acceptable salt is a tartrate, esylate, mesylate, or sulfate salt, preferably mesylate salt.

16. (canceled)

17. The method of claim 1, wherein the pharmaceutical composition is a solid dosage form, oral dosage form and/or tablet form.

18. The method of claim 1, wherein the pharmaceutical composition comprises a 0.5-20.0 mg dose of rasagiline, 0.5-10.0 mg dose of rasagiline, or 0.5-2.0 mg dose of rasagiline.

19. The method of claim 1, wherein the pharmaceutical composition comprises a 0.5 mg dose of rasagiline, 1.0 mg dose of rasagiline, or 2.0 mg dose of rasagiline.

20-24. (canceled)

25. The method of claim 1, wherein determining the genotype of the subject at said one or more SNPs comprises:
(i) obtaining DNA from a sample that has been obtained from the subject;
(ii) optionally amplifying the DNA; and
(iii) subjecting the DNA or the amplified DNA to restriction fragment length polymorphism (RFLP) analysis, sequencing, single strand conformation polymorphism analysis (SSCP), chemical cleavage of mismatch (CCM), gene chip, denaturing high performance liquid chromatography (DHPLC) and polymerase chain reaction (PCR), an array, or a combination thereof.

26. The method of claim 1, wherein the human subject is a naive patient.

27. The method of claim 1, wherein the human subject has been previously administered a Parkinson's disease drug other than rasagiline.

28. The method of claim 2, wherein the genotype of the subject at said one or more SNPs is obtained indirectly by determining the genotype of the subject at a SNP that is in linkage disequilibrium with said one or more SNPs.

29-30. (canceled)

31. A diagnostic kit for evaluating responsiveness to treatment with rasagiline in a human subject afflicted with Parkinson's disease, the kit comprising
(i) at least one probe specific for SNP rs1076560 or rs2283265, and
(ii) instructions for use of the at least one probe to evaluate responsiveness of the subject to treatment with rasagiline;
or
a physical or electronic database comprising the polymorphic profiles of human subjects afflicted with PD, wherein each polymorphic profile includes the diploid genotype of fewer than 10000 SNPs, and the fewer than 10000 SNPs include rs1076560, and rs36023.

32-38. (canceled)

39. A method of determining the identity of the alleles of fewer than 10000 single nucleotide polymorphisms (SNPs) in a subject selected from the group of subjects consisting of human subjects diagnosed with Parkinson's disease to produce a polymorphic profile of the selected subject diagnosed with Parkinson's disease, comprising
(i) obtaining a biological sample comprising a genome from the selected subject diagnosed with Parkinson's disease;
(ii) selecting for allelic identity analysis at least a SNP located at rs1076560 and a SNP located at rs2283265 within the genome of the selected subject diagnosed with Parkinson's disease; and
(iii) assaying, with a probe or a primer, whether
a) the allelic identity at rs1076560 is CC within the nucleotide sequence of the genome in the biological sample of step i), and
b) the allelic identity at rs2283265 is CC within the nucleotide sequence of the genome in the biological sample of step i), and
wherein fewer than 10000 SNPs are selected for allelic identity analysis in step ii) and the same fewer than 10000 SNPs are assayed in step iii).

40-52. (canceled)
